(19) United States
(12) Patent Application Publication
(10) Pub. No.: US 2002/0119940 A1
(43) Pub. Date: Aug. 29, 2002

(54) Gene expression and delivery systems and uses

(76) Inventors: Jeff Nordstrom, College Station, TX (US); Bruce Freimark, Spring, TX (US); Deepa Deshpande, The Woodlands, TX (US)

Correspondence Address: LYON & LYON LLP/ VALENTIS INC., 633 WEST FIFTH STREET, SUITE 4700, LOS ANGELES, CA, 90071-2066, US

(21) Appl. No.: 09/754,014

(22) Filed: Jan. 3, 2001

Related U.S. Application Data
(63) Continuation of application No. 08/948,958, filed on Oct. 10, 1997, now abandoned.
(60) Provisional application No. 60/028,687, filed on Oct. 18, 1996.

(30) Foreign Application Priority Data
Oct. 10, 1997 (US) PCT/US97/18832

(51) Int. Cl. A61K 48/00; C07H 21/04; C12N 15/86

(52) U.S. Cl. 514/44; 536/23.1; 435/320.1; 435/456

(57) ABSTRACT

Plasmid expression systems for delivery of DNA coding sequences to a mammal are described which provide expression of multiple coding sequences from a single plasmid. Also described are particular lipid/DNA delivery systems having advantageous characteristics of size, charge ratio, and proportion of supercoiled DNA, and methods of preparing and using such delivery systems for treatment or as immunization adjuvants.

### RELATED APPLICATIONS

This application relates to U.S. patent application Ser. No. 60/028,687, filed Oct. 18, 1996, entitled “GENE EXPRESSION AND DELIVERY SYSTEMS AND USES” by Nordstrom et al. (Lyon & Lyon Docket No. 221/131) which is incorporated herein by reference in its entirety, including any drawings.

### BACKGROUND OF THE INVENTION

The following discussion of the background and of the invention is provided to aid the reader in understanding the invention and is not admitted to describe or constitute prior art to the present invention.

The present invention relates to gene delivery and gene therapy, and provides novel nucleic acid constructs for expression of eukaryotic genes in a mammal, formulations for delivery that incorporate a nucleic acid construct for expression, and methods for preparing and using such constructs and formulations. In particular, this invention relates to plasmid constructs for delivery of therapeutic nucleic acids to cells and to modulation of cytokine activity. In addition, this invention relates to methods of using those constructs as well as methods for preparing such constructs.

Plasmids are an essential element in genetic engineering and gene therapy. Plasmids are circular DNA molecules that can be introduced into bacterial cells by transformation which replicate autonomously in the cell. Plasmids allow for the amplification of cloned DNA. Some plasmids are present in 20 to 50 copies during cell growth, and after the arrest of protein synthesis, as many as 1000 copies per cell of a plasmid can be generated. (Suzuki et al., Genetic Analysis, p. 404, (1989).)

Current non-viral approaches to human gene therapy require that a potential therapeutic gene be cloned into plasmids. Large quantities of a bacterial host harboring the plasmid may be fermented and the plasmid DNA may be purified for subsequent use. Current human clinical trials using plasmids utilize this approach. (Recombinant DNA Advisory Committee Data Management Report, December, 1994, Human Gene Therapy 6:535-548). Studies normally focus on the therapeutic gene and the elements that control its expression in the patient when designing and constructing gene therapy plasmids. Generally, therapeutic genes and regulatory elements are simply inserted into existing cloning vectors that are convenient and readily available.

Plasmid design and construction utilizes several key factors. First, plasmid replication origins determine plasmid copy number, which affects production yields. Plasmids that replicate to higher copy number can increase plasmid yield from a given volume of culture, but excessive copy number can be deleterious to the bacteria and lead to undesirable effects (Fitzwater, et al., EMBO J. 7:3289-3297 (1988); Uhlin, et al., Mol. Gen. Genet. 165:167-179 (1979)). Artificially constructed plasmids are sometimes unstably maintained, leading to accumulation of plasmid-free cells and reduced production yields.

To overcome this problem of plasmid-free cells, genes that code for antibiotic resistance phenotype are included on the plasmid and antibiotics are added to kill or inhibit plasmid-free cells. Most general purpose cloning vectors contain ampicillin resistance (β-lactamase, or bla) genes. Use of ampicillin can be problematic because of the potential for residual antibiotic in the purified DNA, which can cause an allergic reaction in a treated patient. In addition, β-lactam antibiotics are clinically important for disease treatment. When plasmids containing antibiotic resistance genes are used, the potential exists for the transfer of antibiotic resistance genes to a potential pathogen.

Other studies have used the neo gene which is derived from the bacterial transposon Tn5. The neo gene encodes resistance to kanamycin and neomycin (Smith, Vaccine 12:1515-1519 (1994)). This gene has been used in a number of gene therapy studies, including several human clinical trials (Recombinant DNA Advisory Committee Data Management Report, December, 1994, Human Gene Therapy 6:535-548). Due to the mechanism by which resistance is imparted, residual antibiotic and transmission of the gene to potential pathogens may be less of a problem than for β-lactams.

In addition to elements that affect the behavior of the plasmid within the host bacteria, such as E. coli, plasmid vectors have also been shown to affect transfection and expression in eukaryotic cells. Certain plasmid sequences have been shown to reduce expression of eukaryotic genes in eukaryotic cells when carried in cis (Peterson, et al., Mol. Cell. Biol. 7:1563-1567 (1987); Yoder and Ganesan, Mol. Cell. Biol. 3:956-959 (1983); Lusky and Botchan, Nature 293:79-81 (1981); and Leite, et al., Gene 82:351-356 (1989)). Plasmid sequences also have been shown to fortuitously contain binding sites for transcriptional control proteins (Ghersa, et al., Gene 151:331-332 (1994); Tully and Cidlowski, Biochem. Biophys. Res. Comm. 144:1-10 (1987); and Kushner, et al., Mol. Endocrinol. 8:405-407 (1994)). This can cause inappropriate levels of gene expression in treated patients.

### SUMMARY OF THE INVENTION

The present invention provides compositions and methods for delivery of functional recombinant coding sequences to a mammal. These compositions are prepared and administered in such a manner that the encoded gene products are expressed in the mammal to which the composition is administered. As a result, these compositions and methods are useful in gene therapy since the coding sequence can encode a molecule having a therapeutic function. These compositions include expression systems, delivery systems, and certain coding sequences. The expression systems are constructed to provide the coordinated expression of multiple coding sequences.

The delivery systems are particularly advantageous formulations of a cationic lipid, a co-lipid (preferably a neutral lipid) and a DNA molecule. Such formulations can be administered to a mammal, for example by delivery to a lung, such that one or more coding sequences on the DNA is expressed in that mammal.

Thus, in a first aspect, the invention provides a plasmid for expression of recombinant eukaryotic genes which includes two transcription units. The first transcription unit has a first transcriptional control sequence which is transcriptionally linked with a synthetic 5′ untranslated region, a synthetic intron, a first coding sequence, and a synthetic 3′ untranslated region/poly(A) signal. The second transcription unit has a second transcriptional control sequence which is transcriptionally linked with a synthetic 5′ untranslated region, a synthetic intron, a second coding sequence, and another synthetic 3′ untranslated region/poly(A) signal. It is often advantageous to provide on the plasmid a selectable marker such as an antibiotic resistance gene (e.g., a neomycin resistance gene).

When such a plasmid is placed in an environment suitable for gene expression, the first and second transcriptional units will express both of the encoded gene products. The relative levels of expression of the two gene products will depend to a significant extent on the strength of the associated promoters and the presence and activation of an associated enhancer element. Thus, in a preferred embodiment, the first and/or second transcriptional control sequences include promoter enhancer sequences such as cytomegalovirus (CMV) promotor/enhancer sequences. However, those skilled in the art will recognize that a variety of other promoter sequences suitable for expression in eukaryotic cells are known and can similarly be used in the constructs of this invention.

As used herein, the term “plasmid” refers to a construct made up of genetic material (i.e., nucleic acids). It includes genetic elements arranged such that an inserted coding sequence can be transcribed in eukaryotic cells. Preferably the plasmid also is capable of replication in the eukaryotic host cell, and perhaps in a hon-host cell (e.g., a prokaryotic cell) for production of the plasmid. Also, while the plasmid may include a sequence from a viral nucleic acid, such viral sequence does not cause the incorporation of the plasmid into a viral particle.

The term “expression” refers to the biological production of a product encoded by a coding sequence. In most cases a DNA sequence, including the coding sequence, is transcribed to form a messenger-RNA (mRNA). The messenger-RNA is translated to form a polypeptide product which has a relevant biological activity. However in some cases, an RNA product may have the relevant activity and would thus be regarded as a gene product. Also, the process of expression may involve further processing steps to the RNA product of transcription, such as splicing to remove introns, and/or post-translational processing of a polypeptide product.

The term “transcription unit” refers to a nucleotide sequence which contains at least one coding sequence along with sequence elements which direct the initiation and termination of transcription. A transcription unit may however include additional sequences, which may include sequences involved in post-transcriptional or post-translational processes.

The term “transcriptional control sequence” refers to sequences which control the rate of transcription of a transcriptionally linked coding region. Thus, the term can include elements such as promoters, operators, and enhancers. For a particular transcription unit, the transcriptional control sequences will include at least a promoter sequence. In this context, “transcriptionally linked” means that in a system suitable for transcription, transcription will initiate under the direction of the control sequence(s) and proceed through sequences which are transcriptionally linked with that control sequence(s). Preferably no mutation is created in the resulting transcript, which would alter the resulting translation product.

In the context of this invention in connection with the 5′ untranslated region (UTR), the 3′ UTR/polyA signal, and introns, the term “synthetic” means that the sequence is not initially provided directly from a naturally occurring genetic element of that type but rather is an artificially created sequence, i.e., created by a person by chemical systhesis. A variety of methods for chemical synthesis of nucleic molecules are known to those skilled in the art. After such a synthetic sequence is created, it may be linked with other appropriate nucleotide sequences and replicated and/or transcribed by biological means. While one or more portions of such a synthetic sequence may be the same as portions of naturally occurring sequences, generally the full sequence over the specified genetic element will differ from known naturally occurring genetic element of that type. The use of such synthetic genetic elements allows the functional characteristics of that element to be appropriately designed for the desired function.

Thus, a “synthetic intron” refers to a sequence which is not initally replicated from a naturally occurring intron sequence and which generally will not have a naturally occurring sequence, but which will be removed from an RNA transcript during normal post transcriptional processing. Such introns can be designed to have a variety of different characteristics, in particular such introns can be designed to have a desired strength of splice site and a desired length.

The term “coding region” refers to a nucleic acid sequence which encodes a particular gene product for which expression is desired, according to the normal base pairing and codon usage relationships. Thus, the coding sequence must be placed in such relationship to transcriptional control elements and to translational initiation and termination codons that a proper length transcript will be produced and will result in translation in the appropriate reading frame to produce a functional desired product. Such a coding sequence can be of many different types but preferably encodes a therapeutic molecule or a subunit of such a therapeutic molecule such as an IL-12 subunit.

A “5′ untranslated region” or “5′ UTR” refers to a sequence located 3′ to promotor region and 5′ of the downstream coding region. Thus, such a sequence, while transcribed, is upstream of the translation initiation codon and therefore is not translated into a portion of the polypeptide product. Such a 5′ UTR may have an intron within it.

A “synthetic 3′ untranslated region/poly(A) signal” or “3′ UTR/poly(A) signal” is a sequence located downstream (i.e., 3′) of the region encoding material polypeptide. As with the 5′ UTR this region may be transcribed but not translated. For expression in eukaryotic cells it is generally preferable to include a sequence which signals the addition of a poly-A tail. As with the other synthetic genetic elements the synthetic 3′ UTR is not initially produced directly from a naturally occurring sequence, and generally has a sequence which differs from naturally-occurring UTR elements.

In the genetic constructs of this invention, a first and second synthetic intron, a first and second coding sequence, first and second 5′ UTR, or first and second 3′ UTR may have the same or different first and second elements depending on the desired use or construction efficiency or convenience.

“Cytomegalovirus promotor/enhancer sequences” refers to sequences from a cytomegalovirus which are functional in eukaryotic cells as a transcriptional promoter and an upstream enhancer sequence. The enhancer sequence allows transcription to occur at a higher frequency from the associated promoter.

A “therapeutic molecule” is one which has a pharmacologic activity when administered appropriately to a mammal suffering from a disease or condition. Such a pharmacologic property is one which is expected to be related to a beneficial effect on the course or a symptom of the disease or condition. A “subunit” of a therapeutic molecule is a polypeptide or RNA molecule which combines with one or more other molecules to form a complex having the relevant pharmacologic activity. Examples of such complexes include homodimers and heterodimers as well as complexes having greater numbers of subunits. A specific example of a heterodimer is IL-12, having the p40 and p35 subunits.

The “p40 subunit” is the larger of the two subunits of the IL-12 heterodimer. Thus, it is capable of association with p35 subunit to form a molecule having activities characteristic of IL-12. Human p40 has the amino acid sequence of SEQ ID NO.: 1. Those skilled in the art will recognize that the molecule may have a number of changes from that sequence, such as deletions, insertions or changes of one or a few amino acids, while still retaining IL-12 activity when associated with p35. Such active altered molecules are also regarded as p40.

Conversely, the “p35 subunit” is the smaller of the two heterodimeric subunits of IL-12. For humans p35 has the amino acid sequence of SEQ ID NO.: 5. As for p40, p35 may have a low level of alterations from that sequence while still being regarded as p35.

A particular example of coding regions suitable for use in the plasmids of this invention are the sequences coding for the p40 and p35 subunits of human IL-12. Thus, in a preferred embodiment the first and second coding regions are coding regions for those sequences and are preferably in the order p40 then p35 in the 5′ to 3′ direction.

Thus, a “sequence coding for the p40 subunit of human IL-12” is a nucleic acid sequence which encodes the human p40 subunit as described above, based on the normal base pairing and translational codon usage relationships. The sequence coding for p35 subunit of human IL-12 is similarly defined.

While the above aspect described a plasmid for coordinated expression of two coding regions in separate transcriptional units, the invention also provides plasmids in which such coordinated expression is provided by the use of a plasmid having two coding sequences in a single transcriptional unit. Thus, in a second aspect, the invention provides a plasmid which includes a transcriptional control sequence which is transcriptionally linked with a first coding sequence and a second coding sequence. Also included is a 5′ untranslated region, an intron 5′ to the first coding sequence, and an alternative splice site 3′ to the first coding sequence and 5′ to the second coding sequence, along with a 3′ untranslated region/poly(A) siganl. Such a plasmid provides two different mRNAs. The first results from excision of the intron preceding the first coding sequence during post-transcriptional processing. This mRNA includes both coding regions, however, primarily the first (i.e., 5′) coding region is translated. The second mRNA results from excision of the intron and first coding region by splicing at the alternative splice site. This mRNA thus contains only the second coding region. The strength of the two splice sites can be selected to provide the proper balance of the expression for the two coding sequences.

As in the plasmid described above, various of the specified genetic elements may be synthetic sequences. Also, as in the above plasmid, in preferred embodiments the transcriptional control sequence includes a cytomegalovirus promoter/enhancer sequence. Also in preferred embodiments, the first and second coding sequences are sequences coding for the p40 and p35 subunits of human IL-12.

In this context “alternative splice site” refers to a location along a nucleotide sequence at which the normal intron removal process operates to excise the RNA sequence between a 5′ splice site and a 3′ splice site. The above plasmid contains an intron preceding the first coding sequence which has such 5′ and 3′ splice sites. However, it also contains a second 3′ splice site which is located between the first coding sequence and the second coding sequence. Thus, intron excision can remove either the first intron or the first intron and first coding region. The relative frequencies of these two events depends, in part, on the relative strengths of the two 3′ splice sites, which depends on their precise splice site base sequence.

The invention provides yet another plasmid which provides coordinated expression of two coding sequences within a single transcriptional unit. For this plasmid, the coordinated expression control is provided at the translational level. Thus, in a third aspect the invention provides a plasmid for expression of the recombinant eukaryotic genes which has a transcriptional control sequence transcriptionally linked with a first coding sequence, an IRES sequence, a second coding sequence, a 3′ untranslated region/poly(A) signal, and an intron between the promoter and the first coding sequence. The IRES sequence is between the first coding sequence and the second coding sequence. As in the above aspects, in preferred embodiments the transcriptional control sequence includes a cytomegalovirus promoter enhancer sequence, or the first and second coding sequences include the sequences coding for the p40 and p35 subunits of human IL-12. Also in preferred embodiments the IRES sequence is from an encephalomyocarditis virus. An “IRES sequence” is an internal ribosome entry site. Such IRES sequences have been described in a number of different viruses. Such IRES sequences allow cap independent translation of a coding region 3′ to the IRES sequence.

In additional to plasmids for coordinated expression of multiple coding regions, the invention also provides synthetic coding sequences encoding the human p40 and p35 subunits of human IL-12. Thus, in another aspect the invention provides a synthetic DNA sequence coding for a human IL-12 subunit. Such a synthetic sequence has less than 50% identity to a natural human IL-12 coding sequence. It is preferred that the sequence utilize optimal codon usage; preferably at least 50%, 70%, or 90% of the codons are optimized. Thus, in a preferred embodiment the synthetic DNA sequence has at least 80, 90, 95, or 99% sequence identity to the sequence of SEQ ID NO. 3. In a more preferred embodiment, the DNA includes a sequence identical to the sequence of SEQ ID NO. 3 or 4. These synthetic coding sequences thus encode human p40 IL-12 subunit.

Similarly, in a preferred embodiment, the invention provides a synthetic DNA sequence coding for human p35 IL-12 subunit. The sequence has at least 80, 90, 95, or 99% sequence identity with the sequence of SEQ ID NO. 7. In more preferred embodiments, the synthetic DNA sequence includes a sequence identical to SEQ ID NO. 7 or 8.

For delivery of coding sequences for gene expression, it is generally useful to provide a delivery composition. Such a composition can, for example, aid in maintaining the integrity of the DNA and in enhancing cellular uptake of the DNA. Thus, in another aspect, the invention provides such a composition for delivery of DNA in a mammal, which includes a cationic lipid with a co-lipid, preferably having neutral charge, in which these lipids are prepared as liposomes having an extrusion size of about 800 nanometer (nm), and a quantity of DNA having coding sequences.

As the form of the DNA affects the expression efficiency, it is preferable that a large fraction of the DNA be in supercoiled form. Thus, in preferred embodiments, at least 80, 90, or 95% of the DNA in the composition is supercoiled.

As the charge ratio of the cationic lipid and the DNA is also a significant factor, in preferred embodiments the DNA and the cationic lipid are present is such amounts that the negative to positive charge ratio is about 1:3. While preferable, it is not necessary that the ratio be 1:3. Thus, preferably the negative to positive charge ratio for the compositions is between about 1:1.5 and 1:20, more preferably between about 1:2 and 1:6.

In another preferred embodiment, the composition also includes a carbohydrate solution which is approximately isotonic with mammalian cells. More preferably, the carbohydrate contains about 10% lactose. Also, in a preferred embodiment, the cationic lipid is DOTMA and the neutral co-lipid is cholesterol. DOTMA is described and discussed in Eppstein et al., U.S. Pat. No. 4,897,355, issued Jan. 30, 1990, which is incorporated herein by reference.

The term “cationic lipid” refers to a lipid which has a net positive charge at physiological pH, and preferably carries no negative charges at such pH. An example of such a lipid is DOTMA. Similarly, “neutral co-lipid” refers to a lipid which is uncharged at physiological pH. An example of such a lipid is cholesterol. Generally it is preferred that the co-lipid be neutrally charged, however, charged co-lipids can also be used in some circumstances, particularly lipids with low levels of charge.

Thus, “negative to positive charge ratio” for the DNA and cationic lipid refers to the ratio between the net negative charges on the DNA compared to the net positive charges on the cationic lipid. If the co-lipid carries a charge, that charge is also included in the charge ratio.

In a closely related aspect, the invention provides a composition for delivery of DNA molecules, which includes a cationic lipid with a neutral co-lipid and a quantity of DNA which includes a coding sequence. The DNA and cationic lipid are present in amounts such that the negative to positive charge ratio is about 1:3. Also similar to above, a higher proportion of supercoiled DNA is preferred. Also, preferably, the composition includes an isotonic carbohydrate solution, which is preferably about 10% lactose. Again, in preferred embodiments, the cationic lipid is DOTMA and the neutral co-lipid is cholesterol.

In a related aspect, the invention provides a method for preparing a composition for delivery to a mammal by preparing a DNA which includes a coding sequence to be delivered, preparing liposomes having an extrusion size of about 800 nanometer (nm) which include a cationic lipid and a neutral co-lipid, and combining the liposomes with the DNA in such amounts that the DNA and the cationic lipid are present in a negative to positive charge ratio of about 1:3. Preferably, the coding sequence encodes a therapeutic molecule.

In another aspect, the invention provides a method of treating a mammalian condition or disease. The method involves administering to a mammal suffering from a condition or disease an amount of a composition for delivery of a DNA to a mammal. The composition includes DNA which has coding sequence for a therapeutic molecule, a cationic lipid, and a neutral co-lipid. The DNA and the cationic lipid are present in such amounts as to result in a negative to positive charge ratio of about 1:3. Preferably the composition also contains an isotonic carbohydrate solution, such as an about 10% lactose solution.

For preparing the composition for administration, ultrasonic nebulization provides an effective method to provide an appropriate aerosol. Thus, in a preferred embodiment the composition is prepared for administration by such ultrasonic nebulization.

It is recognized that delivery of a therapeutic coding sequence can be conveniently performed to produce therapeutic effects for a number of specific diseases. Thus, in preferred embodiments the disease or condition is asthma or is a cancer.

As indicated above, human IL-12 is an appropriate molecule for delivery. Therefore, in another preferred embodiment, the DNA includes two coding sequences, one of which encodes human IL-12 p40 subunit, while the other encodes human IL-12 p35 subunit. Compositions including such DNA constructs can be used to treat a variety of diseases, including, for example, asthma and various cancers.

It has also been shown that IL-12 functions as an adjuvant when administered in conjunction with DNA immunization. In this use the IL-12 enhances the immune system effects of the immunization, thereby enhancing the biological response. Therefore, in another aspect, the invention provides a vaccine adjuvant which includes a cationic lipid, a neutral co-lipid, and a DNA. The DNA includes sequences coding for the p40 and p35 subunits of IL-12. The cationic lipid and DNA are present in a negative to positive charge ratio of about 1:3.

The invention further provides a method of enhancing the immunological response of a mammal to a vaccine by administering by administering a vaccine and an adjuvant as described above.

Other features and advantages of the invention will be apparent from the following Description of the Preferred Embodiments and from the claims.

### BRIEF DESCRIPTION OF THE FIGURES

FIG. 1 is a schematic of an expression plasmid showing a single transcription unit. The plasmid includes the bacterial elements, kanamycin resistance gene (kanR) and the plasmid origin of replication. The plasmid also include eukaryotic elements, CMV enhancer/promoter, synthetic intron, synthetic 3′UTR/poly(A) signal, and synthetic 5′ UTR.

FIG. 2 is an expanded schematic showing additional features of an exemplary transcriptional unit incorporating synthetic 5′UTR, synthetic intron, and synthetic 3′UTR/poly(A) signal, as constructed for the exemplary expression systems.

FIG. 3 schematically illustrates the four plasmid construct strategies used for IL-12 expression. Panel A shows a two plasmid arrangement. Panel B shows a plasmid construct having two coding regions with an internal ribosome entry site (IRES) between them. Panel C shows a plasmid construct having alternative RNA splicing, which produces two distinct mRNAs. Panel D shows a plasmid containing two separately transcribed genes. In the schematic diagrams, the transcription start site specified by the CMV enhancer/promoter is indicated by the open arrow, and the poly(A) site is marked by the downward arrow. Each gene contains the synthetic post-transcriptional elements described in FIG. 1, namely, synthetic intron (thin line), synthetic 5′ UTR (black boxes at the 5′ end that are spliced together), and synthetic 3′ UTR/poly(A) signal (black box at the 3′ end). The IRES element in pIN0744 and the alternative 3′ splice site in pIN0745 and pIN0772 are indicated by internal black boxes. Each mRNA is defined by a cap at its 5′ end (m⁷G) and a poly(A) tail (Aₙ) at its 3′ end.

FIGS. 4A-D shows the amino acid sequence of human IL-12 p40 subunit, along with the codons which can code for each of the amino acids.

FIGS. 5A-C shows the amino acid sequence of human IL-12 p35 subunit, along with the codons which can code for each of the amino acids.

FIG. 6 is a table showing the frequencies of codon usage in highly expressed human genes.

FIG. 7 shows the level of secretion of human IL-12 by transfected A549 cells. Human A549 cells in 6-well dishes, 3×105 cells/well, were transfected with 4 μg plasmid formulated with 24 μg lipofectamine. 40 hours post transfection, cell culture supernatants were assayed for human IL-12 heterodimer by ELISA (R&D Systems). pIN0773 represents the two gene construct; a second two gene construct is pIN0774. Cotransfection with two separate plasmids is represented by pIN0728/pIN0755. The alternative splicing construct, pIN0772, is approximately one fourth as effective as pIN0773. The other alternative splicing construct is pIN0745, and the IRES construct is pIN0744.

FIG. 8 shows the expression levels of chloramphenicol acetyltransferase (CAT) from pDNA:Lipid formulations in rat lungs following instillation for assays performed 48 hours after instillation. Two different cationic lipids and two different neutral lipids were utilized, at two different charge ratios (1:3 and 1:0.5 negative to positive). EDOPC:DOPE and DOTMA:Chol with 1:3 charge ratios allowed high level expression. DOPE refers to dioleoylphosphatidylethanolamine, and EDOPC refers to ethyl dioleylphosphatidylcholine.

FIG. 9 illustrates the dose response for CAT expression in rat lungs as a function of DNA amount for instilled exemplary formulations containing 2, 10, or 50 μg of CAT encoding plasmid DNA. As expected, an increase in the amount of DNA delivered resulted in increased expression.

FIG. 10 is a schematic timeline for the Guinea Pig Antigen-Induced Airway Inflammation Model (also referenced as guinea pig asthma model) used for evaluating the effects of the exemplary IL-12 formulations. The timeline shows the days on which ovalbumin (OA) injections and aerosol OA challenge were performed, along with the timing of administration of the IL-12 formulation and measurement of bronchioalveolar lavage (BAL) cell counts.

FIG. 11 is a graph showing the reduction of total BAL cell counts as well as the reduction in eosinophil counts in the BAL cells in response to administration of an IL-12 formulation for three different amounts of administered DNA. The reductions are compared to the effects of the administration of CAT encoding formulations (pCT0129:lipid). The IL-12 formulation contained the exemplary two transcriptional unit plasmid as described.

### DETAILED DESCRIPTION OF THE PREFERRED EMBODIMENTS

### I. General

As described in the Summary above, this invention concerns expression systems for coordinated expression of two or more genes, and formulations and methods for delivering such expression systems or other expression systems to a mammal. In addition, a particular genetic construct is described which includes nucleotide sequences coding for the human IL-12 subunits. Such a construct can beneficially be formulated and administered as described herein, utilizing the expression systems of this invention.

For a number of molecules, it is necessary or beneficial to provide more than one coding sequence. While this can be accomplished by transfecting cells with multiple expression vectors, joint transfection of a cell will occur at a lower frequency than transfection with a single vector. This is particularly important for multi-subunit molecules which require the incorporation of more that one type of component molecules for activity. In common examples, the molecule includes two or more different polypeptide chains, all of which must be associated to produce the relevant biological activity. The difficulty of joint transfection can, however, be eliminated by the use of multi-valent plasmid expression systems.

Joint expression of multiple coding sequences from one plasmid can be accomplished in a number of ways. These include the use of multiple transcriptional units under the control of separate promoters. The relative strengths of the promoters can be selected prior to construction of the plasmid to provide an appropriate balance in expression levels of the different products.

To allow convenient production of such plasmids, it is generally preferable that the plasmid be capable of replication in a cell to high copy number. Generally such production is carried out in prokaryotic cells, particularly including Esherichia coli (E. coli) cells. Thus, the plasmid preferably contains a replication origin functional in a prokaryotic cell and preferably the replication origin is one which will direct replication to a high copy number.

It is also preferable to utilize synthetic genetic elements in the plasmid constructs. As described below, these elements affect post-transcriptional processing in eukaryotic systems. Thus, the use of synthetic sequences allows the design of processing characteristics as desired for the particular application. Commonly, the elements will be designed to provide rapid and accurate processing.

For delivery of DNA encoding a desired expression product to a mammalian system, it is usually preferable to utilize a delivery system. Such a system can provide multiple benefits, notably providing stabilization to protect the integrity of the DNA, as well as assisting in cellular uptake. It has been demonstrated that a cationic lipid:neutral co-lipid mixture (e.g., DOTMA:cholesterol) is effective for such purposes, though other such combinations are also effective. In addition, it has been demonstrated that the manner of preparing the lipid combination and the relative amounts of lipid and DNA present are significant parameters determining the level of expression from the DNA coding regions.

The compositions and methods of the present invention are useful for delivering genes encoding a large variety of products to mammals. Particular examples of proteins that can be encoded by the genes on the plasmid constructs described herein include cytokines and regulatory proteins involved in the immune system.

A large number of regulatory proteins have been identified which are involved in signal transfer between cells of the immune system. Among these are the colony stimulating factors, the interleukins, the interferons, and the tumor necrosis factors.

Interleukin 12 (IL-12) was originally identified as a factor which stimulates natural killer cells and promotes the maturation of cytotoxic T lymphocytes (CTL). IL-12 is a glycoprotein cytokine produced by macrophages and B lymphocytes, which has been shown to have a variety of biological activities involving components of the immune system, in particular T cells and natural killer (NK) cells. Among other effects, it induces production of IFN-γ and TFN from T cells and NK cells, and enhances the cytotoxic activity of those cells. IL-12 has been shown to be a central mediator in the cell-mediated immune response, and therefore has therapeutic use to stimulate that response in a variety of contexts, including microbial and viral infections, certain cancers, and allergic asthma.

IL-12 is a heterodimer composed of p35 and p40 subunits that are linked by a single disulfide bond. For the synthesis of functional IL-12 heterodimers, the pre-polypeptides for p35 and p40 must be synthesized by the same cell. Prepolypeptides are processed by the endoplasmic reticulum/Golgi apparatus, assembled into heterodimers, and secreted as functional molecules. p40 subunits are capable of dimerization. These p40 homodimers bind to IL-12 receptors nonproductively, thereby inhibiting IL-12 function.

### II. Plasmid Construct Expression Systems

### A. Plasmid Design and Construction

For the methods and constructs of this invention, a number of different plasmids were constructed which are useful for delivery and expression of gene sequences, and especially for the coordinate expression of two different coding sequences. Thus, these plasmids contain coding regions for polypeptides which are desired to be expressed, along with genetic elements necessary or useful for expression of those coding regions. In the exemplary embodiments described herein, the coding regions encode the two subunits (p40 and p35 subunits) for human IL-12.

While these embodiments utilized IL-12 cDNA clones from a particular source as described below, those skilled in the art could readily obtain IL-12 coding sequences from other sources, or obtain a coding sequence by identifying a cDNA clone in a library using a probe(s) based on the published IL-12 subunit sequences. In addition, the plasmid constructs of this invention are also suitable for use with a variety of other coding regions, particularly in applications where expression of two coding regions in the same cellular location is desired. Usually, the two coding regions will be different, however, the regions can be the same, thereby providing increased gene dosage.

The source of the coding sequences for human IL-12 p35 and p40 subunits were full length CLMF cDNA clones constructed by Ueli Gubler, as described in Gubler et al., 1991, PNAS 88:4143-4147. These coding sequences include the signal sequences needed for heterodimer formation and secretion. For incorporation into expression plasmids suitable for gene therapy, the coding sequences were PCR amplified using primers with appropriate restriction enzyme sites.

Sequence analysis of clones of the amplified sequences revealed a point mutation in p35. Sequence analysis of the plasmid used as the p35 source revealed the same mutation. This point mutation apparently was introduced during PCR amplification of the original p35 cDNA described in Gubler et al. (1991). The mutation was cured by exchanging a restriction fragment with CLMF 35 Kd Subunit Clone #3, a partial cDNA clone, obtained from Ueli Gubler, that was never subjected to PCR amplification. DNA sequencing verified the integrity of the repair job.

Since the full nucleotide and amino acid sequences of p40 and p35 are known, IL-12 subunit coding sequences can be obtained by a variety of routine methods. For example, coding sequences can be obtained by PCR amplification of genomic or cDNA sequences.

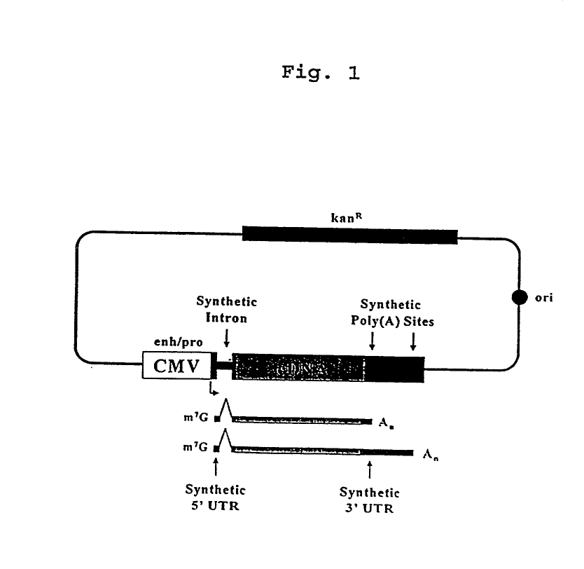

Coding sequences for p35 and p40 were incorporated into an expression plasmid that contains eukaryotic and bacterial genetic elements, as shown schematically in FIG. 1. Eukaryotic genetic elements include the CMV enhancer/promoter, and a combination of post-transcriptional signals (5′ UTR, intron, 3′ UTR/poly(A) signal) that influence gene expression by controlling the accuracy and efficiency of RNA processing, mRNA stability, and translation. All of the post-transcriptional elements are synthetic, derived from synthetic oligonucleotides, and thus are not obtained directly from natural genetic sequences. These synthetic elements are appropriate for use in many different expression vectors, including vectors having only one coding region for delivery, and are therefore not limited to use in the exemplary multi-valent plasmid constructs described herein.

The synthetic intron is designed with splice sites that ensure that RNA splicing is accurate and efficient. The synthetic 3′ UTR/poly(A) signal is designed to facilitate mRNA 3′ end formation and mRNA stability. The synthetic 5′ UTR is designed to facilitate the initiation of translation. The design of the exemplary synthetic elements is described in more detail below.

1. Summary of Synthetic Element Features

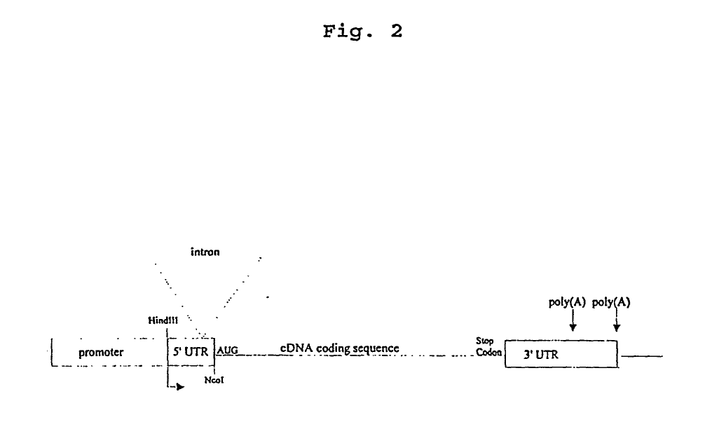

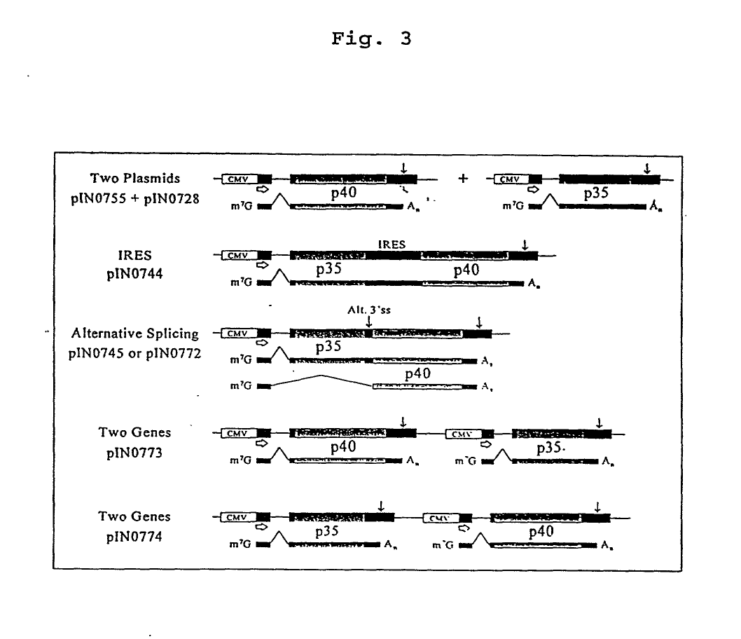

Each of the transcription units in the exemplary two transcription unit plasmid described below (pIN0773) is structured as shown schematically in FIG. 2. The synthetic 5′UTR, intron, and 3′UTR/polyA signal have the general features shown below:

| | |
|---|---|
| 5′ UTR | Short. |
| | Lack of secondary structure. |
| | Kozak sequence. |
| | Site for intron insertion. |
| Intron | 5′ splice site sequence matches |
| | consensus. |
| | 5′ splice site sequence is exactly |
| | complementary to 5′ end of U1 snRNA. |
| | Branch point sequence matches consensus. |
| | Branch point sequence is complementary to |
| | U2 snRNA. |
| | 3′ splice site matches consensus. |
| | Polypyrimidine tract is 16 bases in |
| | length and contains 7 consecutive T's. |
| | (The tract preferably contains at least 5 |
| | consecutive T's.) |
| | Contains internal restriction enzyme |
| | sites. |
| | BbsI cleaves at the 5′ss, EarI cleaves at |
| | the 3′ss. |
| 3′ UTR/Poly(A) | Based on rabbit β-globin 3′ UTR/poly(A) |
| | signal. |
| | Consists of two poly(A) signals in |
| | tandem. |

2. Features of the Synthetic 5′UTR (UT6):

The 5′ untranslated region (5′UTR) influences the translational efficiency of messenger RNA, and is therefore an important determinant of eukaryotic gene expression. The synthetic 5′UTR sequence (UT6) has been designed to maximize the translational efficiency of mRNAs encoded by vectors that express genes of therapeutic interest.

The sequence of the synthetic 5′ UTR (UT6) is shown below. The Kozak sequence is in boldface and the initiation codon is double underlined. The location of the intron (between residues 48 and 49) is indicated by the filled triangle and the sequences that form the exonic portion of consensus splice sites are single underlined. The restriction sites for HindIII and NcoI are overlined.

HindIII ▾ NcoI
AAGCTTACTCAACACAATAACAAACTTACTTACAATCTTAATTAACAGGCCACCATGG

The 5′ untranslated region (5′ UTR), located between the cap site and initiation codon, is known to influence the efficiency of mRNA translation. Any features that influence the accessibility of the 5′ cap structure to initiation factors, the binding and subsequent migration of the 43S preinitiation complex, or the recognition of the initiation codon, will influence mRNA translatability. An efficient 5′ UTR is expected to be one that is moderate in length, devoid of secondary structure, devoid of upstream initiation codons, and has an AUG within an optimal local context (Kozak, 1994, Biochimie 76:815-821; Jansen et al., 1994). A 5′ UTR with these characteristics should allow efficient recognition of the 5′ cap structure, followed by rapid and unimpeded ribosome scanning by the ribosome, thereby facilitating the translation initiation process.

The sequence of the synthetic 5′UTR was designed to be moderate in length (54 nucleotides (nts)), to be deficient in G but rich in C and A residues, to lack an upstream ATG, to place the intended ATG within the context of a optimal Kozak sequence (CCACCATGG), and to lack potential secondary structure. The synthetic 5′ UTR sequence was also designed to lack AU-rich sequences that target mRNAs to be rapidly degraded in the cytoplasm.

Experiments have demonstrated that introns increase gene expression from cDNA vectors, and that introns located in the 5′ UTR are more effective than ones located in the 3′ UTR (Huang and Gorman, 1990, Mol. Cell. Biol. 10:1805-1810; Evans and Scarpulla, 1989, Gene 84:135-142; Brinster et al., 1988, Proc. Natl. Acad. Sci. USA 85:836-840; Palmiter et al., 1991, Proc. Natl. Acad. Sci. USA 88:478-482; Choi et al., 1991, Mol. Cell. Biol. 11:3070-3074). Accordingly, the synthetic 5′ UTR sequence was designed to accommodate an intron with consensus splice site sequences. The intron may, for example, be located between residues 48 and 49 (See intron sequence structure below). The CAG at position 46-48 is the exonic portion of a consensus 5′ splice site. The G at position 49 is the exonic portion of a consensus 3′ splice site.

To facilitate cloning manipulations, the synthetic 5′ UTR sequence was designed to begin with a HindIII site and terminate with a NcoI site.

3. Features of the Synthetic Intron

RNA splicing is required for the expression of most eukaryotic genes. For optimal gene expression, RNA splicing must be highly efficient and accurate. A synthetic intron, termed OPTIVS8B, was designed to be maximally efficient and accurate.

The structure of the exemplary synthetic intron, OPTIVS8 is shown below. Sequences for the 5′ splice site (5′ss), branch point (bp), and 3′ splice site (3′ss) are double underlined. The recognition sequences for the restriction enzymes BbsI and EarI are overlined. The cleavage site for BbsI corresponds to the 5′ss, and the cleavage site for EarI corresponds to the 3′ss.

```
        5′ss bp 3′ss
          | BbsI  | EarI |
5′CAG GTAAGTGTCTTC---(77)---TACTAACGGTTCTTTTTTTCTCTTCACAG G 3′
```

The 5′ splice site (5′ss) sequence matches the established consensus sequence, MAG ↓ GTRAGT, where M=C or A, and R=G or A. Since the mechanism of splicing involves an interaction between the 5′ss of the pre-mRNA and U1 snRNA, the 5′ss sequence of OPTIVS8B was chosen to be exactly complementary to the 5′ end of U1 snRNA.

| | |
|---|---|
| 5′ss | 5′ CAGGUAAGU 3′ |
| | \|\|\|\|\|\|\|\|\| |
| U1 RNA | 3′ GUCCAUUCA 5′ |

In mammals, the consensus sequence for branch points (YNYTRAY, where Y=C or T, R=A or G, N=any base, and the underlined A residue is the actual branch point) is very ambiguous. Since the mechanism of splicing involves an interaction between the branch point (bp) of the pre-mRNA and U2 snRNA, the branch point sequence of OPTIVS8B was chosen to maximize this interaction. (Note that the branch point itself is bulged out). The chosen sequence also matches the branch point sequence that is known to be obligatory for pre-mRNA splicing in yeast. The branch point is typically located 18-38 nts upstream of the 3′ splice site. In OPTIVS8B, the branch point is located 24 nts upstream from the 3′ splice site.

| | |
|---|---|
| BP | 5′ UACUAAC 3′ |
| | \|\|\|\|\| \| |
| U2 RNA | 3′ AUGAU G 5′ |

The sequence of the 3′ splice site (3′ss) matches the established consensus sequence, Y₁₁NYAG ↓ G, where Y=C or T, and N=any base. In 3′ splice sites, the polypyrimidine tract (Y₁₁) is the major determinant of splice site strength. For optimal splice site function in OPTIVS8B, the length of the polypyrimidine tract was extended to 16 bases, and its sequence was adjusted to contain 7 consecutive T residues. This feature was included because Roscigno et al., 1993, J. Biol. Chem. 268:11222-11229, demonstrated that optimal splicing requires the presence of at least 5 consecutive T residues in the polypyrimidine tract.

Splicing in vitro is generally optimal when introns are >80 nts in length (Wieringa, et al., 1984; Ulfendahl et al., 1985, Nucl. Acids Res. 13:6299-6315). Although many introns may be thousands of bases in length, most naturally occurring introns are 90-200 nt in length (Hawkins, 1988, Nucl. Acids Res. 16:9893-9908). The length of the synthetic intron (118 nts) falls within this latter range.

OPTIVS8B was designed with three internal restriction enzyme sites, BbsI, NheI, and EarI. These restriction sites facilitate the screening and identification of genes that contain the synthetic intron sequence. In addition, the BbsI and EarI sites were placed so that their cleavage sites exactly correspond to the 5′ss (BbsI) or 3′ss (EarI). The sequence of the polypyrymidine tract was specifically designed to accommodate the EarI restriction site. Inclusion of the BbsI and EarI sites at these locations is useful because they permit the intron to be precisely deleted from a gene. They also permit the generation of an “intron cassette” that can be inserted at other locations within a gene.

The 77 bases between the BbsI site and the branch point sequence are random in sequence, except for the inclusion of the NheI restriction site.

4. Features of the Synthetic 3′ UTR/poly(A) Signal:

The 3′ ends of eukaryotic mRNAs are formed by the process of polyadenylation. This process involves site specific site RNA cleavage, followed by addition of a poly(A) tail. RNAs that lack a poly(A) tail are highly unstable. Thus, the efficiency of cleavage/polyadenylation is a major determinant of mRNA levels, and thereby, of gene expression levels. 2XPA1 is a synthetic sequence, containing two efficient poly(A) signals, that is designed to be maximally effective in polyadenylation.

A poly(A) signal is required for the formation of the 3′ end of most eukaryotic mRNA. The signal directs two RNA processing reactions: site-specific endonucleolytic cleavage of the RNA transcript, and stepwise addition of adenylates (approximately 250) to the newly generated 3′ end to form the poly(A) tail. A poly(A) signal has three parts: hexanucleotide, cleavage site, and downstream element. The hexanucleotide is typically AAUAAA and cleavage sites are most frequently 3′ to the dinucleotide CA (Sheets et al., 1987). Downstream elements are required for optimal poly(A) signal function and are located downstream of the cleavage site. The sequence requirement for downstream elements is not yet fully established, but is generally viewed as UG- or U-rich sequences (Wickens, 1990; Proudfoot, 1991, Cell 64:671-674; Wahle, 1992, Bioessays 14:113-118; Chen and Nordstrom, 1992, Nucl. Acids Res. 20:2565-2572).

Naturally occurring poly(A) signals are highly variable in their effectiveness (Peterson, 1992). The effectiveness of a particular poly(A) signal is mostly determined by the quality of the downstream element. (Wahle, 1992). In expression vectors designed to express genes of therapeutic interest, it is important to have a poly(A) signal that is as efficient as possible.

Poly(A) efficiency is important for gene expression, because transcripts that fail to be cleaved and polyadenylated are rapidly degraded in the nuclear compartment. In fact, the efficiency of polyadenylation in living cells is difficult to measure, since nonpolyadenylated RNAs are so unstable. In addition to being required for mRNA stability, poly(A) tails contribute to the translatability of mRNA, and may influence other RNA processing reactions such as splicing or RNA transport (Jackson and Standart,1990, Cell 62:15-24; Wahle, 1992).

Some eukaryotic genes have more than one poly(A) site, implying that if the cleavage/polyadenylation reaction fails to occur at the first site, it will occur at one of the later sites. In COS cell transfection experiments, a gene with two strong poly(A) sites yielded approximately two-fold more mRNA than one with a single strong poly(A) site (Bordonaro, 1995). These data suggest that a significant fraction of transcripts remain unprocessed even with a single “efficient” poly(A) signal. Thus, it may be preferable to include more than one poly(A) site.

The sequence of the exemplary synthetic poly(A) signal is shown below. The sequence is named 2XPA. The hexanucleotide sequences and downstream element sequences are double underlined, and the two poly(A) sites are labeled as pA#1 and pA#2. Convenient restriction sites are overlined. The entire 2XPA unit may be transferred in cloning experiments as a XbaI-KpnI fragment. Deletion of the internal BspHI fragment results in the formation of a 1XPA unit.

```
  XbaI BspHI
TCTAGAGCATTTTTCCCTCTGCCAAAAATTATGGGGACATCATGAAGCCCCTTGAGCATCTGACG
                                                              pA#1
                Hex | Downstream element
TCTGGCTAATAAAGGAAATTTATTTTCATTGCAATAGTGTGTTGGAATTTTTTGTGTCTCTCACT
                                                                                      BspHI
CGGTACTAGAGCATTTTTCCCTCTGCCAAAAATTATGGGGACATCATGAAGCCCCTTGAGCATCT
                                                                      pA#2
                        Hex | Downstream element
GACGTCTGGCTAATAAAGGAAATTTATTTTCATTGCAATAGTGTGTTGGAATTTTTTGTGTCTCT
            KpnI
CACTCGGTACC
```

The sequence of the synthetic poly(A) site shown above is based on the sequence of the rabbit β-globin poly(A) signal, a signal that has been characterized in the literature as strong (Gil and Proudfoot, 1987, Cell 49:399-406; Gil and Proudfoot, 1984, Nature 312:473-474). One of its key features is the structure of its downstream element, which contains both UG- and U-rich domains.

A double-stranded DNA sequence corresponding to the 1XPA sequence was constructed from synthetic oligonucleotides. Two copies of the 1XPA sequence were then joined to form the 2XPA sequence. The sequences were joined in such as way as to have a unique XbaI site at the 5′ end of the first poly(A) signal containing fragment, and a unique KpnI site at the 3′ end of the second poly(A) signal containing fragment.

5. Exemplary Plasmid Constructs

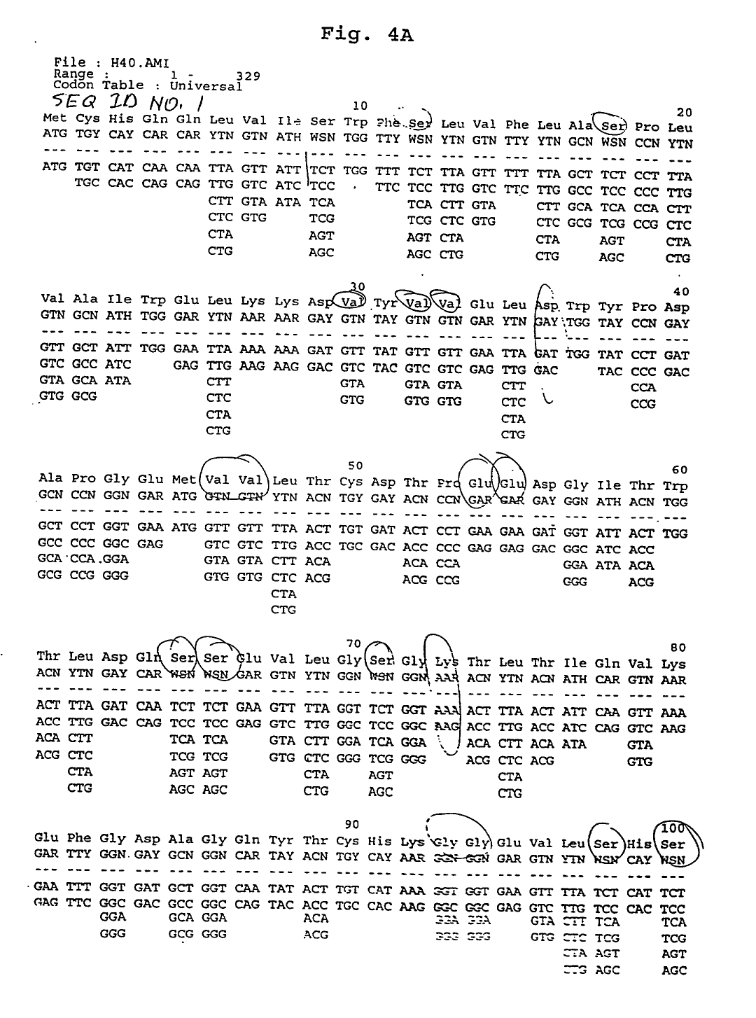

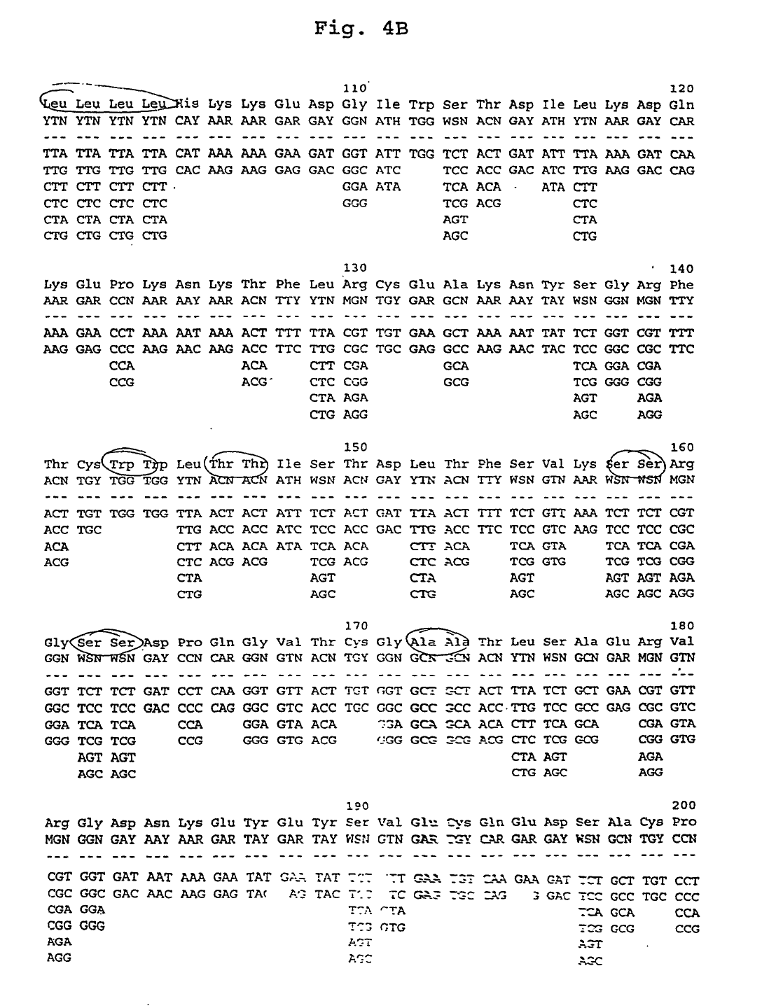

Plasmid constructs for four expression strategies for the joint expression of two coding sequences (e.g., for the subunits of human IL-12) were constructed and tested. Examples for the four strategies are schematically illustrated in FIG. 3), with two possibilities shown for the fourth strategy.

The first strategy involved two expression plasmids, one that encodes p35 (pIN0755), and one that encodes p40 (pIN0728). Thus, cells must be cotransfected with both plasmids to produce IL-12 heterodimer.

The second strategy involved a single plasmid that produces a bicistronic mRNA that contains an IRES (internal ribosome entry site) sequence. The source of the IRES sequence was pCITE2a (Novagen), which contains the encephalomyocarditis virus IRES. Other IRES sequences could similarly be used. In pIN0744, the sequence order is p35-IRES-p40. Thus, p35 is translated in a cap-dependent manner, and p40 is translated in an IRES-dependent manner.

The third strategy involved a single plasmid that encodes two alternative mRNAs that are generated by the splicing of a 5′ splice site to one of two alternative 3′ splice sites. One mRNA encodes p35 and p40, with translation primarily of the p35 sequence. The other mRNA encodes p40. For balanced production of the two mRNAs, it is beneficial to balance the relative strengths of the alternative 3′ splice sites. In pIN0745, both 3′ splice sites have identical sequences, and therefore identical strengths. In pIN0772, the relative strength of the first 3′ splice site was weakened by site-directed mutagenesis to change three consecutive T's to A's. Thus, in the sequence shown above for the OPTIVS8 intron, the included sequence 5′-CTTTTTTTC-3′ was changed to 5′-CTTTAAATC-3′.

The fourth strategy involved a single plasmid that contains two separate transcription units, each driven by its own CMV enhancer/promoter. In pIN0773, the p40 gene is located immediately upstream of the p35 gene. In pIN0774, the order was reversed, such that the p35 gene is located immediately upstream of the p40 gene.

### B. IL-12 Subunit Sequences

The nucleotide sequences of natural human IL-12 subunit coding sequences are known, and are provided below.

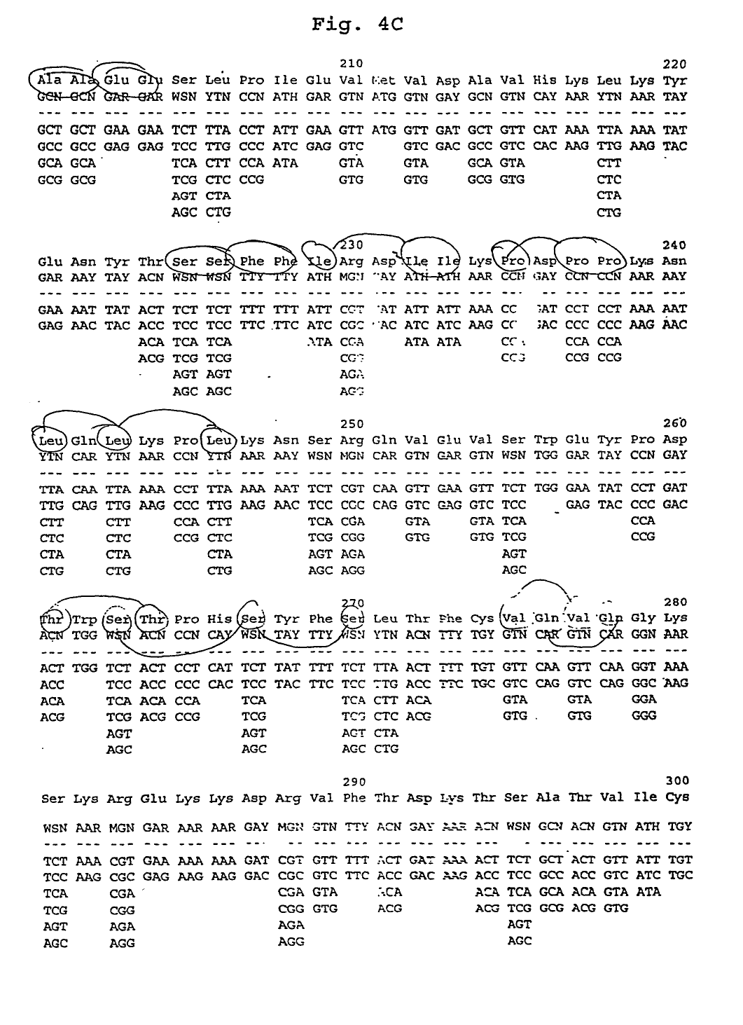

The human p40 subunit is a 306 amino acid polypeptide with a predicted molecular weight of approximately 34.7 kiloDaltons (kd). The amino acid sequence of human IL-12 p40 subunit is shown in FIG. 4 as SEQ ID NO. 1. Sequence Table 1 below contains three nucleotide sequences, each of which encodes human IL-12 p40. The first sequence (SEQ ID NO. 2) is provided by the top rows of each row triplet. This sequence is a natural p40 coding sequence.

Instead of the natural sequences, as provided above, it is advantageous to utilize synthetic sequences which encode the p40 subunit. Such synthetic sequences have alternate codon usage from the natural sequence, and thus have dramatically different nucleotide sequences from the natural sequence. In particular, synthetic sequences can be used which have codon usage at least partially optimized for expression in a human. The natural sequences do not have such optimal codon usage. Preferably, substantially all the codons are optimized.

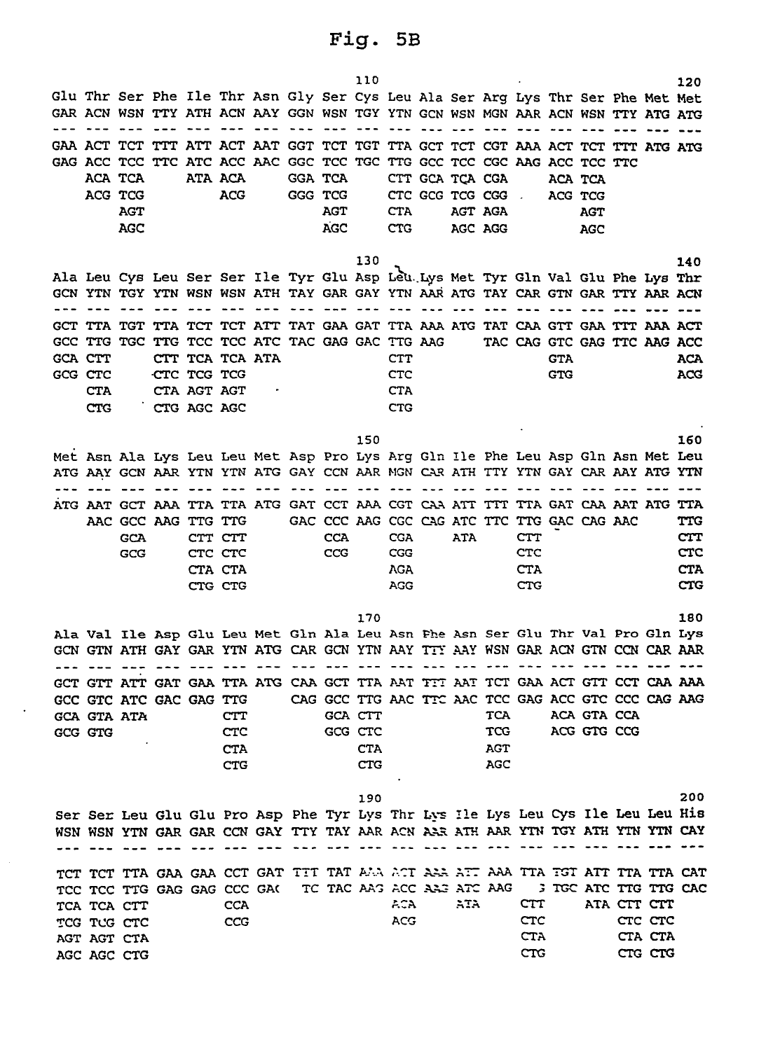

Optimal codon usage in humans is indicated by codon usage frequencies for highly expressed human genes, as shown in FIG. 6. The codon usage chart is from the program “Human_High.cod” from the Wisconsin Sequence Analysis Package, Version 8.1, Genetics Computer Group, Madison, Wis. The codons which are most frequently used in highly expressed human genes are presumptively the optimal codons for expression in human host cells, and thus form the basis for constructing a synthetic coding sequence.

A synthetic sequence encoding the p40 subunit and having optimized codon usage is shown in the second lines of Sequence Table 1 below (SEQ ID NO. 3).

However, rather than a sequence having fully optimized codon usage, it may be desirable to utilize p35 and p40 encoding sequences which have optimized codon usage except in areas where the same amino acid is too close together or abundant to make uniform codon usage optimal. Thus, the third rows in Sequence Table 1 below provides a synthetic p40 encoding sequence (SEQ ID NO. 4) which has optimal codon usage except for such changes to reduce codon uniformity in particular regions of the sequence.

Sequence TABLE 1
Sequences Encoding Human IL-12 p40

```
First line = natural sequence (SEQ ID NO. 2)
Second line = all codons optimized (SEQ ID NO. 3)
Third line = all codons optimized except when same amino
          acids were too close/abundant (changes between
          second and third lines bolded) (SEQ ID NO. 4)
ATG TGT CAC CAG CAG TTG GTC ATC TCT TGG TTT TCC CTG GTT TTT CTG GCA TCT CCC CTC
ATG TGC CAC CAG CAG CTG GTG ATC AGC TGG TTC AGC CTG GTG TTC CTG GCC AGC CCC CTG
ATG TGC CAC CAG CAG CTG GTG ATC AGC TGG TTC TCC CTG GTG TTT CTG GCC AGC CCC CTC
GTG GCC ATA TGG GAA CTG AAG AAA GAT GTT TAT GTC GTA GAA TTG GAT TGG TAT CCG GAT
GTG GCC ATC TGG GAG CTG AAG AAG GAC GTG TAC GTG GTG GAG CTG GAC TGG TAC CCC GAC
GTG GCC ATC TGG GAG CTG AAG AAA GAC GTG TAC GTG GTC GAG CTG GAC TGG TAC CCC GAC
GCC CCT GGA GAA ATG GTG GTC CTC ACC TGT GAC ACC CCT GAA GAA GAT GGT ATC AGC TGG
GCC CCC GGC GAG ATG GTG GTG CTG ACC TGC GAC ACC CCC GAG GAG GAC GGC ATC ACC TGG
GCC CCC GGC GAG ATG GTG GTC CTG ACC TGC GAC ACC CCC GAG GAA GAC GGC ATC ACC TGG
ACC TTG GAC CAG AGC AGT GAG GTC TTA GGC TCT GGC AAA ACC CTG ACC ATC CAA GTC AAA
ACC CTG GAC CAG AGC AGC GAG GTG CTG GGC AGC GGC AAG ACC CTG ACC ATC CAG GTG AAG
ACC CTG GAC CAG AGC AGT GAG GTG CTG GGC TCC GGC AAG ACC CTG ACC ATC CAG GTG AAG
GAG TTT GGA GAT GCT GGC CAG TAC ACC TGT CAC AAA GGA GGC GAG GTT CTA AGC CAT TCG
GAG TTC GGC GAC GCC GGC CAG TAC ACC TGC CAC AAG GGC GGC GAG GTG CTG AGC CAC AGC
GAG TTC GGC GAC GCC GGC CAG TAC ACC TGC CAC AAG GGA GGC GAG GTG CTG AGC CAC TCC
CTC CTG CTG CTT CAC AAA AAG GAA GAT GGA ATT TGG TCC ACT GAT ATT TTA AAG GAC CAG
CTC CTG CTG CTG CAC AAG AAG GAG GAC GGC ATC TGG AGC ACC GAC ATC CTG AAG GAC CAG
CTC CTG CTG CTC CAC AAA AAG GAG GAC GGC ATC TGG AGC ACC GAC ATC CTG AAG GAC CAG
AAA GAA CCC AAA AAT AAG ACC TTT CTA AGA TGC GAG GCC AAG AAT TAT TCT GGA CGT TTC
AAG GAG CCC AAG AAC AAG ACC TTC CTG CGC TGC GAG GCC AAG AAC TAC AGC GGC CGC TTC
AAG GAG CCC AAG AAC AAG ACC TTC CTG CGC TGC GAG GCC AAG AAC TAC AGC GGC CGC TTC
ACC TGC TGG TGG CTG ACG ACA ATC AGT ACT GAT TTG ACA TTC AGT GTC AAA AGC AGC AGA
ACC TGC TGG TGG CTG ACC ACC ATC AGC ACC GAC CTG ACC TTC AGC GTG AAG AGC AGC AGG
ACC TGC TGG TGG CTG ACC ACG ATC AGC ACC GAC CTG ACC TTC AGT GTG AAG AGC AGC AGG
GGC TCT TCT GAC CCC CAA GGG GTG ACG TGC GGA GCT GCT ACA CTC TCT GCA GAG AGA GTC
GGC AGC AGC GAC CCC CAG GGC GTG ACC TGC GGC GCC GCC ACC CTG AGC GCC GAG CGC GTG
GGC TCC AGC GAC CCC CAG GGC GTG ACC TGC GGC GCT GCC ACC CTG AGC GCC GAG CGC GTG
AGA GGG GAC AAC AAG GAG TAT GAG TAC TCA GTG GAG TGC CAG GAG GAC AGT GCC TGC CCA
CGC GGC GAC AAC AAG GAG TAC GAG TAC AGC GTG GAG TGC CAG GAG GAC AGC GCC TGC CCC
CGC GGC GAC AAC AAG GAG TAC GAG TAC AGC GTG GAG TGC CAG GAA GAC TCC GCC TGC CCC
GCT GCT GAG GAG AGT CTG CCC ATT GAG GTC ATG GTG GAT GCC GTT CAC AAG CTC AAG TAT
GCC GCC GAG GAG AGC CTG CCC ATC GAG GTG ATG GTG GAC GCC GTC CAC AAG CTG AAG TAC
GCC GCT GAG GAG AGC CTG CCC ATC GAG GTG ATG GTG GAC GCC GTT CAC AAG CTG AAG TAC
GAA AAC TAC ACC AGC AGC TTC TTC ATC AGG GAC ATC ATC AAA CCT GAC CCA CCC AAG AAC
GAG AAC TAC ACC AGC AGC TTC TTC ATC CGC GAC ATC ATC AAG CCC GAC CCC CCC AAG AAC
GAG AAC TAC ACC AGC AGC TTC TTC ATC CGC GAC ATC ATC AAG CCT GAC CCA CCC AAG AAC
TTG CAG CTG AAG CCA TTA AAG AAT TCT CGG CAG GTG GAG GTC AGC TGG GAG TAC CCT GAC
CTG CAG CTG AAG CCC CTG AAG AAC AGC CGC CAG GTG GAG GTG AGC TGG GAG TAC CCC GAC
CTC CAG CTG AAG CCC CTC AAG AAC TCC CGC CAG GTG GAG GTG AGC TGG GAG TAC CCC GAC
ACC TGG AGT ACT CCA CAT TCC TAC TTC TCC CTG ACA TTC TGC GTT CAG GTC CAG GGC AAG
ACC TGG AGC ACC CCC CAC AGC TAC TTC AGC CTG ACC TTC TGC GTG CAG GTG CAG GGC AAG
ACC TGG AGC ACG CCC CAC TCC TAC TTC TCC CTG ACC TTC TGC GTG CAG GTC CAG GGC AAG
AGC AAG AGA GAA AAG AAA GAT AGA GTC TTC ACG GAC AAG ACC TCA GCC ACG GTC ATC TGC
AGC AAG CGC GAG AAG AAG GAC CGC GTG TTC ACC GAC AAG ACC AGC GCC ACC GTG ATC TGC
AGC AAG CGC GAG AAG AAA GAC CGG GTG TTC ACC GAC AAG ACC AGC GCC ACC GTC ATC TGC
CGC AAA AAT GCC AGC ATT AGC GTG CGG GCC CAG GAC CGC TAC TAT AGC TCA TCT TGG AGC
CGC AAG AAC GCC AGC ATC AGC GTG CGC GCC CAG GAC CGC TAC TAC AGC AGC AGG TGG AGC
CGC AAG AAC GCC AGC ATC AGC GTG CGC GCC CAG GAC CGC TAC TAT AGC TCC TCT TGG AGC
GAA TGG GCA TCT GTG CCC TGC AGT TAG
GAG TGG GCC AGC GTG CCC TGC AGC TAG
GAG TGG GCC AGC GTG CCC TGC TCC TAG
```

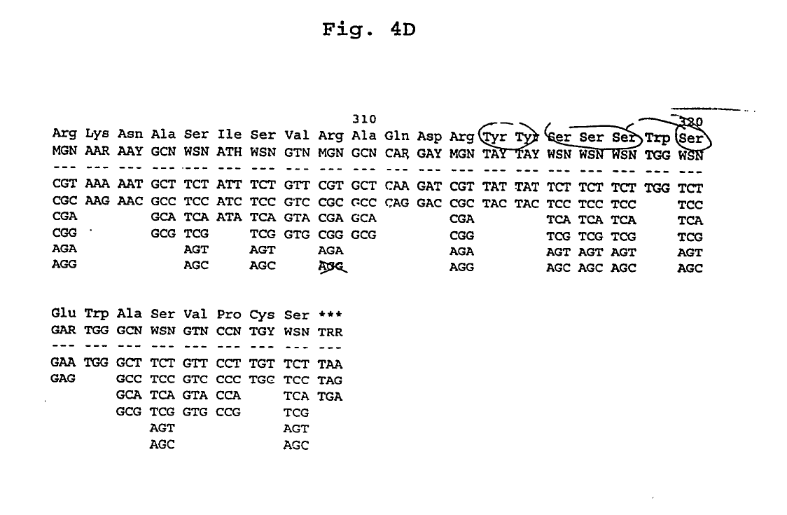

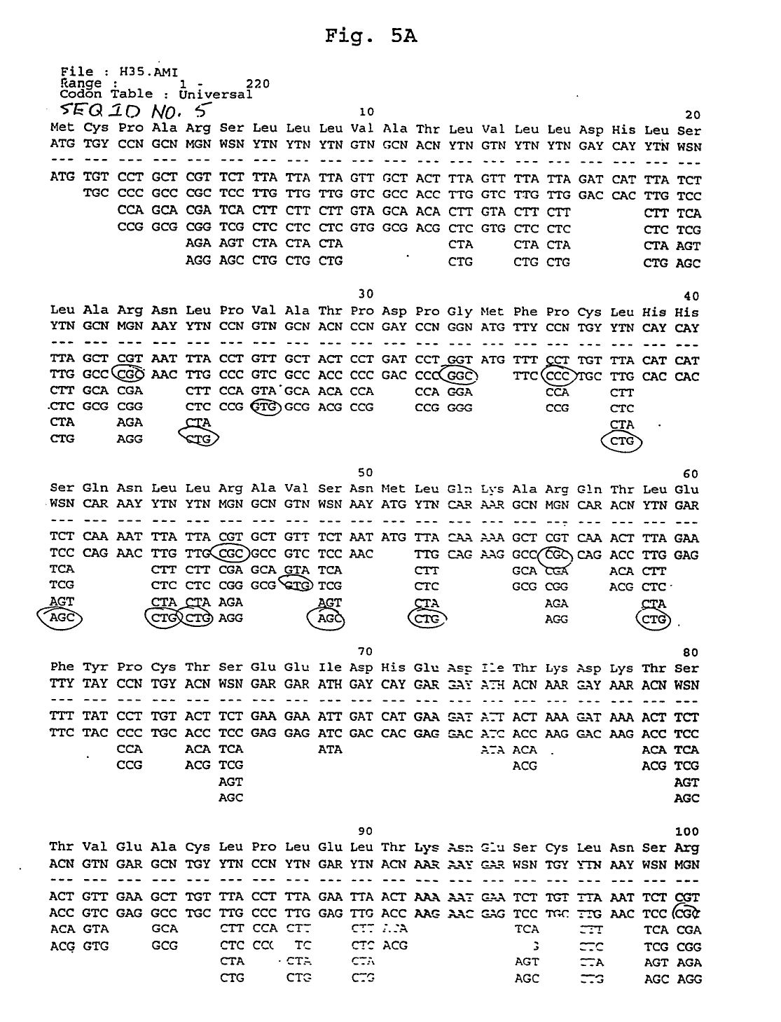

The human p35 subunit is a 197 amino acid polypeptide with a predicted molecular weight of approximately 22.5 kd, the amino acid sequence of which is shown in FIG. 5 as SEQ ID NO. 5. Similarly to Sequence Table 1 above, Sequence Table 2 provides 3 sequences encoding human IL-12 p35 subunit.

As above, the top lines show a natural p35 coding sequence (SEQ ID NO. 6). The second lines show the sequence coding for p35, but having optimal codon usage (SEQ ID NO. 7). The third lines show a sequence coding for p35, having optimal codon usage except where the same amino acids were too close together or too abundant for uniform codon usage (SEQ ID NO. 8).

Sequence TABLE 2
Sequences Encoding Human IL-12 p35

```
First line = natural sequence (SEQ ID NO. 6)
Second line = all codons optimized (SEQ ID NO. 7)
Third line = all codons optimized except when same amino
          acids were too close/abundant (changes between
          second and third lines bolded) (SEQ ID NO. 8)
ATG TGT CCA GCG CGC AGC CTC CTC CTT GTG GCT ACC CTG GTC CTC CTG GAC CAC CTC ACT
ATG TGC CCC GCC CGC AGC CTG CTG CTG GTG GCC ACC CTG CTG CTG CTG GAC CAC CTG AGC
ATG TGC CCC GCC CGC AGC CTG CTG CTC GTG GCC ACC CTG CTG CTC CTG GAC CAC CTC AGC
TTG GCC AGA AAC CTC CCC GTG GCC ACT CCA GAC CCA GGA ATG TTC CCA TGC CTT CAC CAC
CTG GCC CGC AAC CTG CCC GTG GCC ACC CCC GAC CCC GGC ATG TTC CCC TGC CTG CAC CAC
CTG GCC CGC AAC CTC CCC GTG GCC ACC CCA GAC CCC GGC ATG TTC CCA TGC CTG CAC CAC
TCC CAA AAC CTG CTG AGG GCC GTC AGC AAC ATG CTC CAG AAG GCC AGA CAA ACT CTA GAA
AGC CAG AAC CTG CTG GCG GCC GTG AGC AAC ATG CTG CAG AAG GCC GCG CAG ACC CTG GAG
AGC CAG AAC CTG CTG GCG GCC GTG AGC AAC ATG CTG CAG AAG GCC GCG CAG ACC CTG GAG
TTT TAC CCT TGC ACT TCT GAA GAG ATT GAT CAT GAA GAT ATC ACA AAA GAT AAA ACC AGC
TTC TAC CCC TGC ACC AGC GAG GAG ATC GAC CAC GAG GAC ATC ACC AAG GAC AAG ACC AGC
TTC TAC CCC TGC ACC AGC GAG GAG ATC GAC CAC GAG GAC ATC ACC AAG GAC AAG ACC AGC
ACA GTG GAG GCC TGT TTA CCA TTG GAA TTA ACC AAG AAT GAG AGT TGC CTA AAT TCC AGA
ACC GTG GAG GCC TGC CTG CCC CTG GAG CTG ACC AAG AAC GAG AGC TGC CTG AAC AGC CGC
ACC GTG GAG GCC TGC CTG CCC CTC GAG TTA ACC AAG AAC GAG AGC TGC CTC AAC AGC CGC
GAG ACC TCT TTC ATA ACT AAT GGG AGT TGC CTG GCC TCC AGA AAG ACC TCT TTT ATG ATG
GAG ACC AGC TTC ATC ACC AAC GGC AGC TGC CTG GCC AGC CGC AAG ACC AGC TTC ATG ATG
GAG ACC TCC TTC ATC ACC AAC GGC ACT TGC CTG GCC TCC CGC AAG ACC AGC TTC ATG ATG
GCC CTG TGC CTT AGT AGT ATT TAT GAA GAC TTG AAG ATG TAC CAG GTG GAG TTC AAG ACC
GCC CTG TGC CTG AGC AGC ATC TAC GAG GAC CTG AAG ATG TAC CAG GTG GAG TTC AAG ACC
GCC CTG TGC CTG AGC TCC ATC TAC GAG GAC CTG AAG ATG TAC CAG GTG GAG TTC AAG ACC
ATG AAT GCA AAG CTT CTG ATG GAT CCT AAG AGG CAG ATC TTT CTA GAT CAA AAC ATG CTG
ATG AAC GCC AAG CTG CTG ATG GAC CCC AAG CTC CAG ATC TTC CTG GAC CAG AAC ATG CTG
ATG AAC GCC AAG CTC CTG ATG GAC CCC AAG CTC CAG ATC TTC CTG GAC CAG AAC ATG CTG
GCA GTT ATT GAT GAG CTG ATG CAG GCC CTG AAT TTC AAC AGT GAG ACT GTG CCA CAA AAA
GCC GTG ATC GAC GAG CTG ATG CAG GCC CTG AAC TTC AAC AGC GAG ACC GTG CCC CAG AAG
GCC GTG ATC GAC GAG CTG ATG CAG GCC CTG AAC TTC AAC AGC GAG ACC GTG CCC CAG AAG
TCC TCC CTT GAA GAA CCG GAT TTT TAT AAA ACT AAA ATC AAG CTC TGC ATA CTT CTT CAT
AGC AGC CTG GAG GAG CCC GAC TTC TAC AAG ACC AAG ATC AAG CTG TGC ATC CTG CTG CAC
AGC AGC CTG GAG GAG CCC GAC TTC TAC AAG ACC AAG ATC AAG CTG TGC ATC CTG CTG CAC
GCT TTC AGA ATT CGG GCA GTG ACT ATA GAC AGA GTG ACG AGC TAT CTG AAT GCT TCC TAA
GCC TTC CGC ATC CGC GCC GTG ACC ATC GAC CGC GTG ACC AGC TAC CTG AAC GCC ACC TGA
GCC TTC CGC ATC CGG GCC GTG ACC ATC GAC CGC GTG ACC AGC TAC CTG AAC GCC ACG TGA
```

In addition, other synthetic sequences can be used which have substantial portions of the codon usage optimized, for example, with at least 50%, 70%, 80% or 90% optimized codons. Other particular synthetic sequences for the p40 and p35 subunits can be selected by reference to the codon usage chart in FIG. 6. A sequence is selected by choosing a codon for each of the amino acids of the polypeptide sequences. DNA molecules corresponding to each of the polypeptides can then by constructed by routine chemical synthesis methods. For example, shorter oligonucleotides can be synthesized, and then ligated in the appropriate relationships to construct the full-length coding sequences.

### C. In Vitro Data

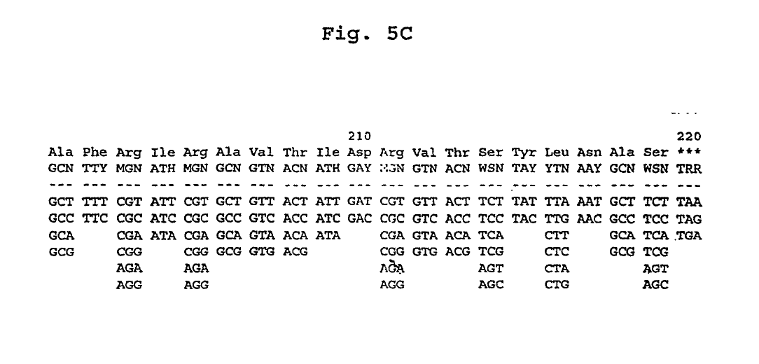

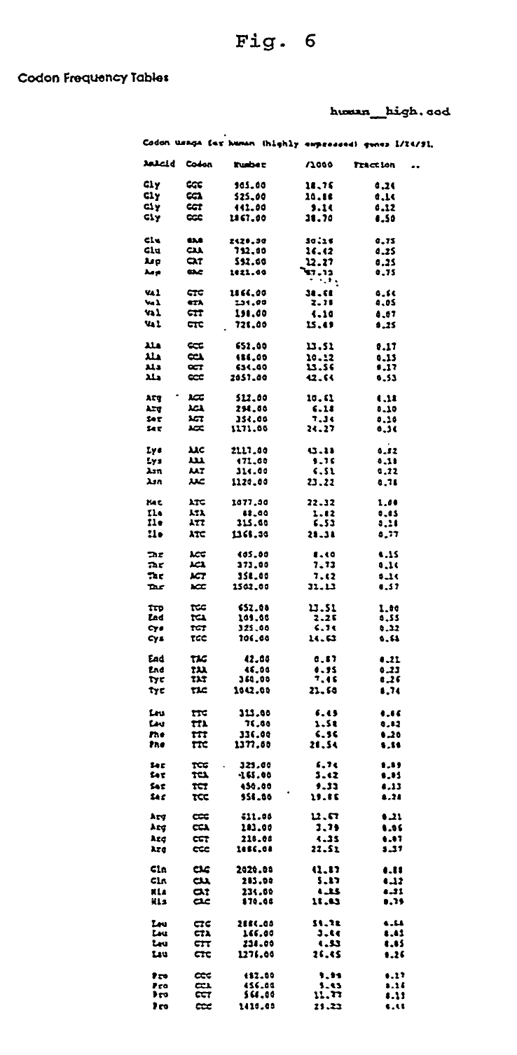

The multivalent expression systems described above were evaluated for their ability to produce IL-12 by transfection into A549 (human lung carcinoma) cells in vitro. Secretion of human IL-12 heterodimer into the cell culture supernatant was assayed by an ELISA method using the QUANTIKINE™ system (R&D Systems). However, any sensitive detection method for IL-12 can be used for such assays. Representative data of two experiments are shown in FIG. 7. IL-12 mRNAs were analyzed by Northern Blot Analysis. RNA was isolated from transfected A549 cells and analyzed by Northern blotting using a 1:1 mixture of radiolabeled p35 and p40 sequences as probe. Because each expression plasmid has two poly(A) sites that are 135 nucleotides apart, each mRNA class is a doublet of two mRNAs that differ by 135 nucleotides. All bands revealed by Northern blot analysis correspond to the expected mRNA sizes. In cells transfected with pIN0773 or cotransfected with pIN0728/pIN0755, p40 mRNA is 1340/1475 nucleotides, and p35 mRNA is 1014/1149 nucleotides. In cells transfected with pIN0744, the bicistronic mRNA is 2511/2646 nucleotides. In cells transfected with pIN0745 or pIN0772, p35 mRNA is 2041/2176 nucleotides, and p40 mRNA is 1341/1476 nucleotides.

The highest levels of expression (800-1000 ng/10⁶ cells/40 h) is obtained with pIN0773, a two gene system, and with pIN0728/pIN0755, the two plasmid system. Northern blot analysis shows that mRNAs of the correct size are formed, and that the accumulation and relative proportions of the p35 and p40 mRNAs is similar with these two systems. In both systems, the level of p40 mRNA is greater than that of p35 mRNA. Since the p35 and p40 genes are identical, except for the nature of the coding sequences, the difference in levels of mRNA accumulation probably reflect differences in mRNA stability. Because of its high level of IL-12 expression as a single plasmid, pIN0773 was selected as the construct for in vivo evaluation.

The second highest level of expression (˜400 ng/10⁶ cells/40 h) was obtained with pIN0774. This plasmid differs from pIN0773 only in the order of the p35 and p40 genes. The RNA profile from Northern blotting is essentially the same as that of pIN0773. Thus, the order of the genes in the two gene plasmids may be a significant variable.

The third highest level of expression (˜200 ng/10⁶ cells/40 h) was obtained with the pIN0772, an alternative splicing construct. pIN0772 has splice sites that were designed to give balanced production of p35 and p40 mRNAs. Northern blot analysis shows that balanced mRNA production was achieved; however, overall mRNA accumulation was reduced. Further optimization of the combination of splice sites can be performed to provide balanced, high level mRNA production.

Lower levels of expression were observed with pIN0745, the other alternative RNA splicing construct. Since the alternative 3′ splice sites of pIN0745 are identical in sequence, RNA splicing is expected to be imbalanced. Northern blot data show high levels of p35 mRNA, but quite low levels of p40 mRNA. Cotransfection of pIN0745 with pIN0728 (encoding the p40 subunit) generates high levels of IL-12. This confirms that pIN0745 is limited in p40 synthesis. This construct design could be improved by altering the relative strengths of the alternative splice sites to provide additional p40 synthesis.

Gene expression was also observed with pIN0744, the IRES-containing construct. Northern blot data shows the presence of high levels of the expected mRNA. Cotransfection experiments with pIN0728 or pIN0755 indicate that pIN0744 is limited in p40 synthesis.

As noted above, pIN0773 is useful for two polypeptide expression. ELISA experiments (FIG. 7) show that it produces high levels of secreted IL-12 in cultured cells (˜1 μg/10⁶ cells/40 h). Northern blot experiments (as described above) indicate that it produces p35 and p40 mRNAs at high level and with the expected sizes. It is contained within a single plasmid.

RT-PCR analysis was performed to evaluate the accuracy of the splicing of the p35 and p40 RNA transcripts. RNA from pIN0773 transfected cells was analyzed by RT-PCR with primers that span the synthetic intron. For the analysis of the p40 mRNA, the upstream primer was in the 5′ UTR, the downstream primer was in the p40 coding sequence. For the analysis of the p35 mRNA, the upstream primer was in the 5′ UTR, the downstream primer was in the p35 coding sequence. For controls, plasmid DNA (P) was analyzed. For p40 mRNA analysis, pIN0728 was used as the plasmid control. For p35 mRNA analysis, pIN0755 was used as the plasmid control. The amplified sequences were analyzed by electrophoresis with 100 bp markers also provided. Comparison of the resulting electrophoresis gel data shows that splicing of each of the RNAs proceeds at the expected locations.

### D. In Vivo Data

Plasmid constructs as described above were incorporated in delivery formulations. Generally, 50 μg of plasmid DNA was combined with DOTMA:chol (equimolar DOTMA:cholesterol prepared as 800 nm liposomes in 10% lactose) with the DNA and DOTMA in a 1:3 negative to positive charge ratio. The formulations were delivered to rat lung by intratracheal instillation. The formulation, delivery, assay, and rat lung model system is described in more detail below.

Comparisons of IL-12 expression after intratracheal instillation of pIN0773, pIN0744, pIN0745, pIN0744/pIN0728, and pIN0745/pIN0728 in rat lung was performed. 50 μg of IL-12 expression plasmids were formulated with DOTMA:Chol 1:3 −/+ and administered to rat lungs by instillation. Lung tissue was harvested 48 hours after instillation and assayed for the level of human IL-12 heterodimer. pIN0773 yielded the highest and most consistent levels of IL-12 expression for the plasmid constructs tested (approximately 800 pg IL-12/lung). Next was the pIN0744/pIN0728 co-transfection, then the pIN0745/pIN0728 co-transfection, followed by the pIN0744 and pIN0745 plasmids.

Thus, pIN0773 exhibited the highest activity of the described constructs in vivo. The higher in vivo activity of pIN0773 over that of pIN0728/pIN0744 and pIN0728/pIN0745 is expected, since delivery of a single multivalent plasmid ensures expression of both subunits within the same cell, whereas delivery of two separate plasmids does not.

Certain of the IL-12 coding constructs were also evaluated in instillation of normal guinea pig lungs, with delivery formulations as described for rat lung instillation except containing 80 μg plasmid DNA. Control formulations containing CAT encoding plasmid DNA, and control formulations containing no plasmid DNA were also provided. The results demonstrated that cotransfection with p0728 and p0744 or p0745 provided greatly enhanced IL-12 expression over transfection only with p0744 or p0745. Similar results were observed both for lung tissue and for bronchioalveolar lavage fluid.

### III. Formulations for Gene Delivery

### A. General

While expression systems such as those described above provide the potential for expression when delivered to an appropriate location, it is beneficial to provide the expression system construct(s) in a delivery system which can assist both the delivery and the cellular uptake of the construct. Thus, this invention also provides particular formulations which include one or more expression system constructs (e.g., DNA plasmids as described above), a cationic lipid, a co-lipid (preferably a neutral co-lipid), and a carbohydrate agent to make the formulation iso-osmotic and isotonic.

Generally, the cationic lipid and the neutral co-lipid in an aqueous-based carbohydrate solution are formed into liposomes, such as by forcing the lipid and aqueous solution through a membrane with pores of a desired size. The liposomes are combined with the DNA to form a DNA/lipid complex, which can then be administered to a mammal by a delivery method appropriate to the desired delivery site. As described below, at least for delivery to the lung, the diameter of the liposomes, and the DNA:cationic lipid charge ratio are significant parameters in determining the resulting level of expression.

An additional significant factor relating to the plasmid construct is the percentage of plasmids which are in a supercoiled (SC) form rather than the open circular (OC) form. In addition, for delivery to the lung, the mode of delivery also affects the resulting expression level. In contrast, the expression level was shown to not significantly depend on the particular plasmid preparation utilized.

### B. Liposome Extrusion Size, DNA:Cationic Lipid (−:+) Charge Ratio, and Percent Supercoiled Plasmid

As indicated above, the parameters of liposome extrusion size, charge ratio, and percent supercoiled plasmid were significant parameters in determining the level of expression observed when the formulated complexes were delivered to the lung. (Lung delivery modes and the effects of different modes on observed expression levels are discussed below.)

For evaluating these parameters, a standard rat lung model system was generally utilized. The system determined the level of expression of chloramphenicol acetyltransferase (CAT) in rat lung using instillation. The rats weighed 180-200 grams. The formulations generally contained DOTMA and cholesterol (chol)as the cationic lipid and neutral co-lipid respectively, along with 10% lactose to make the formulation approximately isotonic to the intracellular fluid. However, similar results are obtained using an alternate cationic lipid, neutral co-lipid combination, thus the formulations are not limited to using DOTMA/chol. The cationic lipid and the neutral co-lipid are preferably present in approximately equimolar amounts.

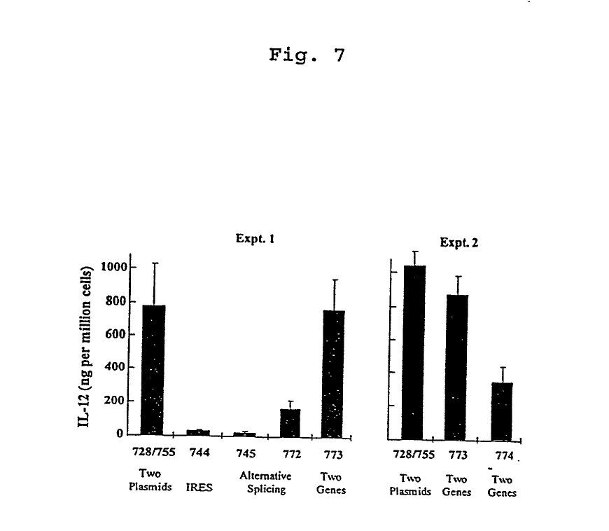

Certain alternative lipid combinations were evaluated which utilized ethyl dioleylphosphatidylcholine (EDOPC) as the cationic lipid and DOPE as the co-lipid. EDOPC is a phospholipid with an ester linker bond. DOPE is dioleoylphosphatidylethanolamine. It was found that EDOPC:DOPE containing formulations prepared from liposomes in the same manner as the DOTMA:Chol containing formulations produced similar levels of expression in rat lung following instillation as found for the DOTMA:Chol formulations. See FIG. 8.

A variety of other cationic lipids and co-lipids are described in Eppstein et al, U.S. Pat. No. 4,897,355 (including DOTMA and DOPE), and in PCT Application No. PCT/US95/03635, International Publication WO 95/26356 (including EDMPC), both of which are incorporated by reference. Thus, other lipid selections can be utilized in the delivery formulations, within the scope of this invention.

Except for tests to demonstrate the effect of the charge ratio on expression level, the DNA and cationic lipid were present in relative amounts such that the negative to positive charge ratio was 1:3. For rats, the formulation was generally administered as a 400 μl volume which contained 50 μg of plasmid DNA. For experiments in which the amount of DNA was varied, the DNA concentration was altered while maintaining the 400 μl instillation volume. The formulation was administered by gavage needle instillation into the trachea of anesthetized, intubated rats.

Expression levels of CAT were determined 48 hours after instillation. Whole lungs were removed into tubes, snap-frozen, and bead beated. The concentration of CAT in the resulting fluid was then determined using a commercial ELISA assay system (CAT ELISA from Boehringer Mannheim). Other assay systems, such as radiolabel or other enzyme-based CAT detection systems could alternatively be used. The CAT ELISA system is a sandwich ELISA method which utilizes immobilized anti-CAT antibodies to bind CAT from a test solution, digoxigenin-labeled anti-CAT antibodies then complex with the immobilized CAT. Peroxidase conjugated anti-digoxigenin then binds to the immobilized complex and reacts with a substrate to give a calorimetric report which can be compared to a calibration curve to provide the CAT concentration in the test sample.

For evaluating the effect of liposome extrusion size, liposomes were prepared by extrusion through a porous membrane having pores of defined sizes. Following preparation of the liposomes, the DNA was added to provide the delivery system complex. The relative amounts of the components were as described above. In one set of tests, liposomes were prepared having three different extrusion sizes, 100, 400, and 800 nm (pore size of the membrane). The mean CAT expression levels increased in the order 100, 400, and 800 nm extrusion size. The mean expression levels were approximately 2000, 11000, and 14000 pg CAT/rat lung respectively. Thus, for the formulations described herein, it is preferable, but not necessary, that an extrusion size of about 800 nm be used for liposome preparation. If the complexes are prepared by a method other than liposome extrusion, it is desirable that the resulting complexes be approximately the size which results from the use of 800 nm liposome extrusion.

The effect of the relative levels of supercoiled and open circular plasmid DNA was evaluated using formulations as above, for which the liposomes were prepared with an extrusion size of about 800 nm. A preparation in which the DNA was primarily supercoiled (SC) (about 80% supercoiled) provided higher CAT expression levels than preparations having primarily open circular (OC) DNA or a combination of SC and OC DNA. The OC tests provided a mean CAT expression level of approximately 1000 pg/lung, while the mixed OC/SC tests provided approximately 2500 pg/lung, and the SC tests provided approximately 6500 pg/lung. Thus, it is shown that generally the expression level increases with increasing SC content, thus, a high level of supercoiling is preferred (e.g., at least 70, 80, 85, 90, or 95% SC).

The effect of the DNA:cationic lipid (−:+) charge ratio was evaluated using formulations having extrusions sizes of about 400 nm. See FIG. 8. The CAT expression level results demonstrated that a negative to positive charge ratio of 1:3 provided higher expression than a charge ratio of 1:0.5. Thus, it is preferable that the formulation used be about 1:3, however other charge ratios, both higher and lower than 1:3, can also provide useful expression.

### C. Delivery and Expression in Lung

A variety of delivery methods can be used with the constructs and formulations described above, in particular, delivery to the lung can be performed using a number of different methods to create and direct the formulation. The first method utilized was intubation and instillation as described above for the analyses of CAT expression. The other modes involved nebulization of the formulation to create an aerosol, which was then directed to the lung. The combinations of nebulization and application techniques included: (1)jet nebulization/breathed fog in exposure chamber; (2)ultrasonic nebulization/intubation; (3)ultrasonic nebulization/mechanical ventilator; and (4)direct nebulization and direction using catheter nebulizer. Most analyses were based on CAT assays, however, some tests used expression systems encoding IL-12.

### 1. Intratracheal Instillation of DNA:Lipid Complex

Instillation of the formulations in rats was performed as generally described above. In addition to the tests already described, dose response analyses were performed for both CAT encoding formulations and IL-12 encoding formulations. These dose response analyses utilized formulations having plasmid DNA with DOTMA/chol and 10% lactose, with a 1:3 negative to positive charge ratio.

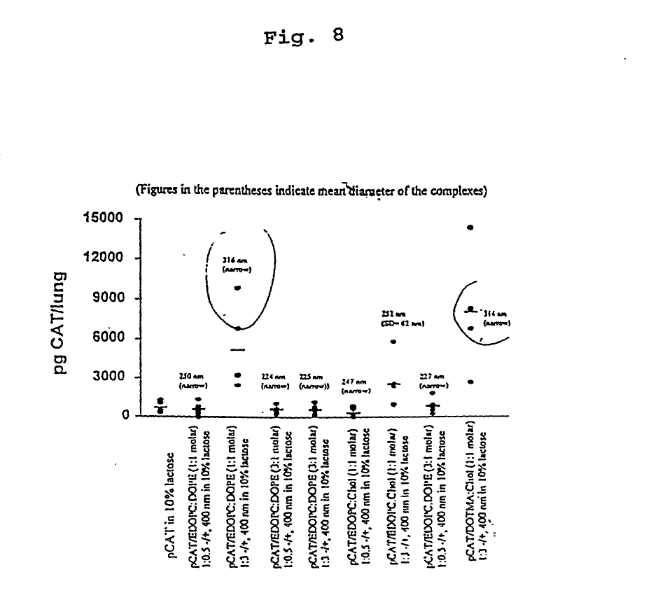

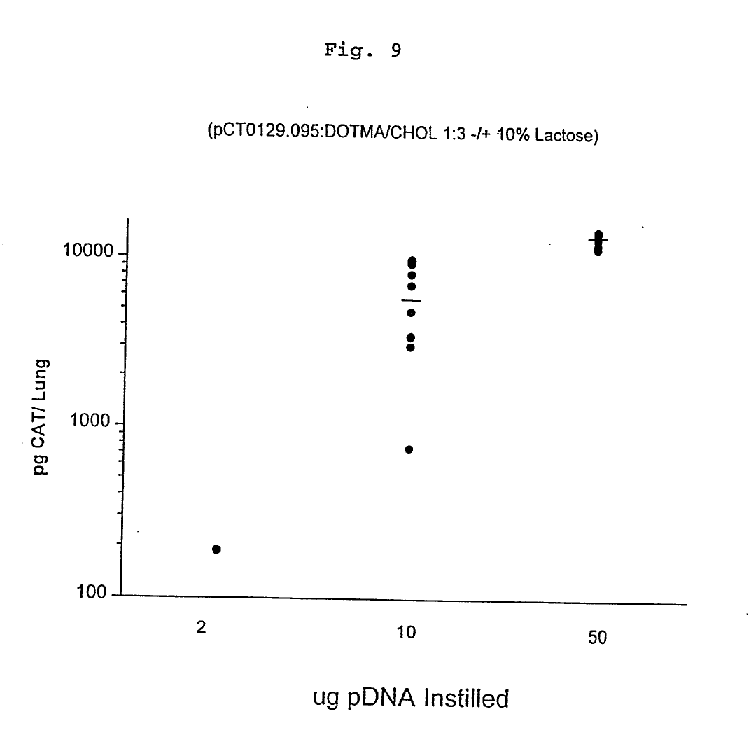

The dose response results for CAT expression is shown in FIG. 9. The amount of CAT in the rat lungs was determined for 3 different amounts of instilled plasmid DNA (2, 10, and 50 μg DNA). The graph suggests an approximately linear dose response over the tested range of DNA amounts.

Similar dose response determinations were performed for IL-12 expression in rat lungs as a function of DNA amount for instilled exemplary formulations containing IL-12 encoding plasmid DNA (using plasmid pIN0773 described below). The formulations contained Dotma/Chol in a 1:1 mole ratio prepared as 800 nm extrusion size liposomes in aqueous 10% lactose, and had a 1:3 negative to positive charge ratio. The amount of IL-12 per rat lung was determined for 4 different amounts of instilled plasmid DNA (3.13, 6.25, 12.5, and 25 μg DNA). The amount of IL-12 increased with increasing instilled plasmid DNA, but the response appeared to be non-linear over the tested range. With 6.25 μg of plasmid DNA, approximately 50 picograms (pg) IL-12 per rat lung was detected. With 12.5 μg of DNA, approximately 350 pg IL-12/lung was detected, and with 25 μg DNA, approximately 1700 pg IL-12/lung was detected.

These results indicate that, as expected, the amount of DNA encoding the desired product delivered to the lungs significantly affects the expression levels obtained.

### 2. a. Ultrasonic Nebulization of DNA:Lipid Complex

This example illustrates the generation and characterization of an aerosol complex.

The plasmid/lipid complexes described in Example 1 were aerosolized according to the manufacturer's instructions using an ultrasonic nebulizer (Model NE-U07, Omron Health Care, Inc., Lake View, Ill.). Aerosolized complexes were collected using a modified test tube impaction apparatus. In this system, the aerosols were fed into a flexible tygon tubing and through a narrow glass pipet. The aerosols that exited the pipet impacted on the ice-cooled test tube and condensed. Aerosols were collected at predetermined time intervals for characterization, as described below.

Stability of the sonic nebulized plasmid/lipid complexes and the DNA within the complexes were assessed using dynamic light scattering and Doppler electrophoretic light scattering as described above. The complexation efficiency and plasmid integrity were determined by agarose gel electrophoresis. For plasmid integrity determinations, the DNA was stripped from the complex by treatment with Triton-X prior to electrophoresis. The structure of the DNA bands in the Triton-treated samples was compared to that of a naked DNA control.

The fraction of supercoiled plasmid in the unnebulized and nebulized complexes was similar to that in the “naked DNA” control. Supercoiled form is the most potent and fragile of plasmid physical forms. The fact that the integrity of the supercoiled form was maintained after nebulization indicates that cationic lipids aid in protecting the DNA from shear induced during droplet formation.

The emitted dose and aerodynamic diameter were determined using standard methods as defined in United States Pharmacopeia<601>. Aerosols were collected on a 0.2 μm filter at a pre-determined flow rate of 3 L/min using a critical flow orifice (CFO). The filter containing the aerosols was washed with 5 mL of 5% sodium dodecyl sulfate (SDS) buffer to separate the DNA from lipids. The solution was centrifuged and assayed spectrophotometrically at a wavelength of 260 nm for DNA concentration. The DNA concentration in the output aerosol stream from the ultrasonic nebulizer was 5 μg/mL.

Aerosols generated from the ultrasonic nebulizer were characterized based on mass median aerodynamic diameter (MMAD) and geometric standard deviation (GSD) using inertial impaction techniques. An Andersen 1 SCFM (28.3 L/min) non-viable ambient sampler, consisting of eight impaction stages and a preseparator, was employed to collect aerosol particles. The aerosols were collected for 5 minutes. Aerosols were collected at each of the eight impaction stages on stainless steel discs and on a glass fiber filter (Gelman Type A/E, Gelman Sciences Inc., Ann Arbor, Mich.) with pore diameter of 0.2 μm. Each of the discs was removed from the impactor, placed in a petri dish and washed with 5 mL of 5% SDS. Each petri dish was shaken at periodic intervals for complete dissolution of the lipid from the deposited particles. The solution was centrifuged and assayed spectrophotometrically at a wavelength of 260 nm for DNA concentration. The cumulative mass fraction of DNA collected on each stage of the cascade impactor was plotted against the effective cut-off diameter for that stage on logarithmic probability paper and a log-normal distribution was calculated for the data by the method of least squares. The MMAD (taken as the point on the regression which equally divided the mass) was 2.4 μm. The GSD (calculated by dividing the particle size below which 84.1% of the distribution by mass occurs by the mass median size) was 3.2. The size distribution of the aerosols indicated that the majority of the particles are in the respirable range.

Tests were performed to compare the level of transgene expression following intratracheal instillation of unnebulized and nebulized plasmid/lipid complexes.

Animals were divided into three treatment groups (three animals/group) and anesthetized with 80 mg/kg of ketamine given intraperitonealy. The treatment groups were intubated with a tracheal catheter and placed supine above an operating table. Aerosols were delivered through the tracheal catheter and carried by the ventilating air stream. Animals were exposed to the aerosolized complex for pre-determined time intervals. Following inhalation, the animals were extubated and allowed to recover from the anesthetic and returned to the animal housing facility. The animals were euthanized by CO₂ asphyxiation 48 hours post-inhalation using a dry ice chamber and the lung tissues were harvested. The tissues were homogenized in Tris/HCl buffer and centrifuged. The supernatant was subsequently analyzed using the ELISA assay for CAT expression according to the manufacturer's instructions (Boehringer Mannheim CAT ELISA Kit, Catalog Number 1363727).

The levels of CAT expression in animals instilled by intratracheal intubation with 400 μl of the ultrasonic nebulized complex (after 10 minutes, collected on an impinger) or residual complex aliquoted after 30 minutes from the nebulizer reservoir (100 μg of plasmid DNA). The results show that the level of transgene expression following intratracheal instillation of unnebulized, nebulized or residual plasmid/lipid complexes was are comparable.

### b. Lung Delivery of Ultrasonic Aerosolized DNA:Lipid Complex Using a Mechanical Ventilator

To increase the mass of aerosolized DNA:lipid complex deposited into the animal's lung the feasibility of using a mechanical ventilator in conjunction with a ultrasonic nebulizer was assessed. The mechanical ventilator is basically a positive volume pump consisting of a cylinder and piston. A positive mechanical side valve was activated by the cam on the motor synchronized inspiration and expiration with the motion of the piston. The respiratory parameters (breathing frequency, tidal volume and, exhalation time) of the animal were controlled by the mechanical ventilator. The ventilator consisted of three ports. The aerosols from the ultrasonic nebulizer was drawn through the bottom port during the backward stroke of the piston. The aerosols were delivered to the animal during the forward piston stroke through the middle port via an tracheal catheter. The exhaled aerosols were vented through the top port.

The following plasmid/lipid complexes and plasmid were formulated in lactose (10% w/v).

CMV-CAT/DOTMA:Chol (800 nm), Charge ratio (−:+) 1:3

Plasmid DNA Concentration: 200 μg/mL

In this study aerosol generated by the ultrasonic nebulizer was delivered to the animal at a rate of 5 mL per breath. The ventilator was operated at a frequency of 100 breaths/min. The duration of exposure was 5 minutes.

There was significant level of CAT expression following inhalation of aerosolized DNA:lipid complex. The level of CAT expression observed in this study was comparable to those obtained following intratracheal instillation. A significant fraction of the aerosolized complex condensed in the aerosol inlet tube and the aerosol delivery tube hooked to the tracheal catheter. In subsequent studies such condensation was minimized by operating the ventilator at a lower frequency.

In those subsequent studies aerosols generated by the ultrasonic nebulizer were delivered to the animal at a rate of 6 mL per breath. The ventilator was operated at a frequencies of 50 and 75 breaths/min. The duration of exposure was increased to 7.5 and 15 minutes, respectively.

There was minimally detectable level of expression using the modified operating procedure. The results from this study suggest that the use of a mechanical ventilator may not be an appropriate approach for increasing the inhaled dose. In order to increase the mass of aerosolized DNA:lipid complex deposited into the lungs, alternate strategies are preferable. Such alternatives include intubation with ultrasonic nebulization and use of a nebulization catheter for aerosol delivery.

### c. Lung Delivery of Ultrasonic Aerosolized DNA:Lipid Complex Using Intubation

Expression levels of CAT in rat lung were determined using a formulation including plasmid DNA encoding CAT with DOTMA:chol prepared as 800 nm extrusion size liposomes. The negative to positive charge ratio was 1:3 for the DNA and cationic lipid. The formulation was ultrasonic nebulized, and delivered to the rat lung by direct intubation. Thus, this method provides both effective aerosol formation and efficient delivery to the application site. Forty eight hours after administration, the amount of CAT present per lung was determined using the CAT ELISA kit from Boehringer Mannheim as described above.

As compared to breathed fog delivery in an exposure chamber, the direct intubation provided much faster complex deposition. In a calculation based on the relative deposition rates for FITC labeled dextran, the ultrasonic nebulization/intubation approach deposited more than 60 times as much volume in a 10 minute exposure as the jet nebulization/breathed fog approach. This enhanced deposition rate is also reflected in the CAT expression levels. The CAT levels after a 10 minute exposure using the ultrasonic nebulization/intubation approach produced similar CAT expression levels as a 240 minute jet nebulization/breathed fog exposure.

### 4. Catheter Nebulization

The nebulization catheter uses a liquid feed in conjunction with a compressed air supply to generate aerosols at the catheter tip. The catheter is about 0.2 to 1.0 mm in diameter and consists of several integral gas and liquid capillaries. These capillaries converge and terminate as tiny orifices at the distal tip of the catheter. Gas and liquid flows through the respective capillaries and exit through the orifices. The intimate contact between the gas and the liquid produces aerosol at the distal tip. The liquid can be pulsed in coordination with the inspiratory phase of the ventilator or delivered manually via a syringe. The catheter can be placed in the airways for targeted inter-pulmonary application of therapeutic aerosols. In vivo studies have shown that the catheter delivers over 95% of the nebulized drug and approximately 85-95% of the aerosolized drug deposits in the lungs. The mass median aerodynamic diameter (MMAD) of the aerosols generated by this device is greater than 5 μm. This size range may be appropriate for intercorporeal nebulization.

CMV-CAT/DOTMA:Chol (800 nm) complexes formulated at a charge ratio (−:+) of 1:3 were aerosolized using the nebulization catheter. The volume of the formulation nebulized was 1 mL. The aerosolized DNA:lipid complex was collected in an impinger. The particle size distribution of the complex before and after nebulization was determined using dynamic light scattering techniques. The DNA concentration in the collected aerosols was quantified using spectrophotometric techniques. The stability of DNA: lipid complex and integrity of the plasmid was assessed using gel electrophoresis.

Gel electrophoresis indicated that the DNA: lipid complex before and after nebulization was stable and integrity of the plasmid was maintained. There was no change in the size distribution of complex before and after nebulization indicating that the colloidal properties of the complex was maintained. The concentration of the DNA in the control and aerosolized formulation were quite similar, suggesting that the device has a high aerosol delivery efficiency.

### D. Storage Stability of IL-12 Formulations

IL-12 formulations as described above have been shown to be stable for at least 4 weeks. The stability of the formulated complex before and after lyophilization was measured for up to 8 weeks.

There was no change in the size of the DNA:lipid complex from week 0 through 8. This observation indicates that the colloidal properties of the complex were maintained in the wet and lyophilized formulations. Also the fraction of supercoiled form of the plasmid in the complex showed no statistically significant differences from week 0 through 8. This observation suggests that the integrity of the plasmid was maintained upon storage.

In view of these observations, it is expected that formulations containing DNA encoding other products will exhibit similar stability.

### IV. Pharmacologic Activity of Exogenous IL-12

The administration of exogenous IL-12 has been shown to have pharmacological activity in a number of animal models. The results observed for the administration of IL-12 in these models is indicative of therapeutic potential IL-12 in humans. Such results include demonstrations of the effects of IL-12 in mouse model of asthma and the effects on mouse tumors of various types, both directly and as an adjuvant to DNA immunization.

### A. Effects of Exogenous IL-12 on Allergen-Induced Airway Changes (Allergic Asthma)

Examples of reports of IL-12 effects in animal asthma models is provided in Kips et al., 1995, Int. Erch. Allergy. Immunol. 107:115-118, and Kips et al. 1996, Am. J. Respir. Crit. Care Med. 153:535-539. These references describe the effects of IL-12 on antigen induced airway changes in mice. In this model the mice were actively sensitized to ovalbumin (OA) by intraperitoneal injection. Then on days 14-21 post injection the animals were exposed to aerosolized ovalbumin in an exposure chamber. This regime resulted in airway eosinophilia, production of ovalbumin specific IgE, and airway hyperresponsiveness to carbachol. Administration of IL-12 by injection on days 0-5 significantly reduced the allergen induced influx of eosinophils detected in BAL fluid and inhibited allergen induced IgE synthesis, as well as abolishing the hyperresponsiveness to carbachol. It was also shown in this model that administration of IL-12 along with the aerosol ovalbumin exposure on days 14-21 eliminated the airway eosinophilia and hyperresponsiveness despite the presence of circulating specific IgE.

These results, along with demonstration of the effects of IL-12 administration in mice infected with Nippostrongylus brasiliensis, suggest that IL-12 can be an effective therapeutic agent for certain immune system related disorders.

### B. Anti-Tumor Activity of IL-12

Administration of IL-12 has also been shown to be effective in causing the regression, eradication, or prevention of establishment of various tumors in mice. While antitumor effects have been demonstrated using a systemic administration of IL-12 (Brenda et al., 1993, J. Ex. Med. 178:1223; Nastala et al., 1994, J. Immunol.), it has also been shown that IL-12 gene therapy is effective.

One such demonstration is reported in Rakhmilevich et al., 1996, Proc. Natl. Acad. Sci. USA, 93:6291-6296. Using particle mediated (gene gun) in vivo delivery, the effects of the expression of an exogenous IL-12 gene on a number of different intradermal tumors was investigated. It was demonstrated that, for Renca, MethA, SA-1, and L5178Y implanted tumors, delivery of the IL-12 cDNA into epidermal cells overlaying the tumor resulted in detectable levels of the IL-12 protein at the treatment site and in complete regression of the tumor. Complete regression required 1-4 treatments beginning on day 7 after tumor implantation.

It was further shown, using a metastatic P815 tumor model, that delivery of IL-12 cDNA into the skin over an advanced intradermal tumor, followed by excision of the tumor and three additional IL-12 treatments, reduced the occurrence of systemic metastatic tumors. This reduction resulted in prolonged survival of the test mice. It was also shown that the tumor regression resulting from IL-12 treatment was also associated with the development of a memory immune response against the specific tumor.

Thus, it was shown that IL-12 in vivo gene therapy is an effective approach for eradication or suppression of intradermal tumors in a variety of mouse tumor models. These results suggest that IL-12 gene therapy in humans will have similar therapeutic effects.

Likewise in Tehara et al., 1996, J. Immunol., the effects of IL-12 gene therapy on intradermal mouse tumors was studied. Tehara et al. utilized a retroviral expression vector encoding the IL-12 subunits. Using the MCA207 methylcholanthrene induced sarcoma, it was shown that implanted cells transfected with the IL-12 encoding retroviral vector failed to produce tumors. In contrast, non-tranfected cells and cells transfected with the retroviral vector not encoding IL-12 consistently produced palpable tumors.

Also it was shown that inoculation of a mouse in one flank with IL-12 transfected cells inhibited the formation of tumors from non-transfected cells inoculated in the other flank. Strong inhibition was reported when the inoculations were performed at the same time, however, significant inhibition occurred even when the IL-12 tranfected cells were inoculated up to three days after the non-tranfected cells. As with the Rakhmilevich study a tumor specific memory immune response was also demonstrated.

As indicated by the above references, reported results demonstrate that antitumor therapy using exogenous IL-12, whether systemically administered or administered through gene therapy, is effective in a variety of mouse tumor models. Likewise, such studies suggest that IL-12 therapy will be an effective antitumor treatment in humans.

### C. Adjuvant Effects of IL-12

In addition to the use of IL-12 as a direct agent in therapeutic models for asthma and various tumors, it has also been shown that IL-12 will function as an adjuvant to enhance the response in antitumor DNA immunization. For example, in Irvine et al., 1996, J. Immunol. 156:238-245, the study used a mouse adenocarcinoma cell line tranfected with a β-galactosidase gene. The β-galactosidase served as a model antigen. DNA immunization using gene gun delivery of a plasmid encoding β-galactosidase prevented the growth of metastatic tumors from a subsequent tumor cell challenge. However, this immunization had little or no effect on the growth of established metastatic tumors.

In contrast, treatment including administration of IL-12 to mice bearing such established tumors 18-24 hours following DNA immunization provided a substantial reduction in the number of pulmonary metastases as compared to DNA immunization alone or IL-12 administration alone. Thus, the subsequent administration of IL-12 provided an adjuvant effect, enhancing the DNA immunization.

An adjuvant effect by IL-12 has also been demonstrated with a number of other antigens. Such results suggest that antitumor DNA immunization enhanced with an IL-12 adjuvant can be a useful antitumor therapy.

### V. Effects of Delivery of IL-12 Formulations in Guinea Pig Antigen-Induced Airway Inflammation Model

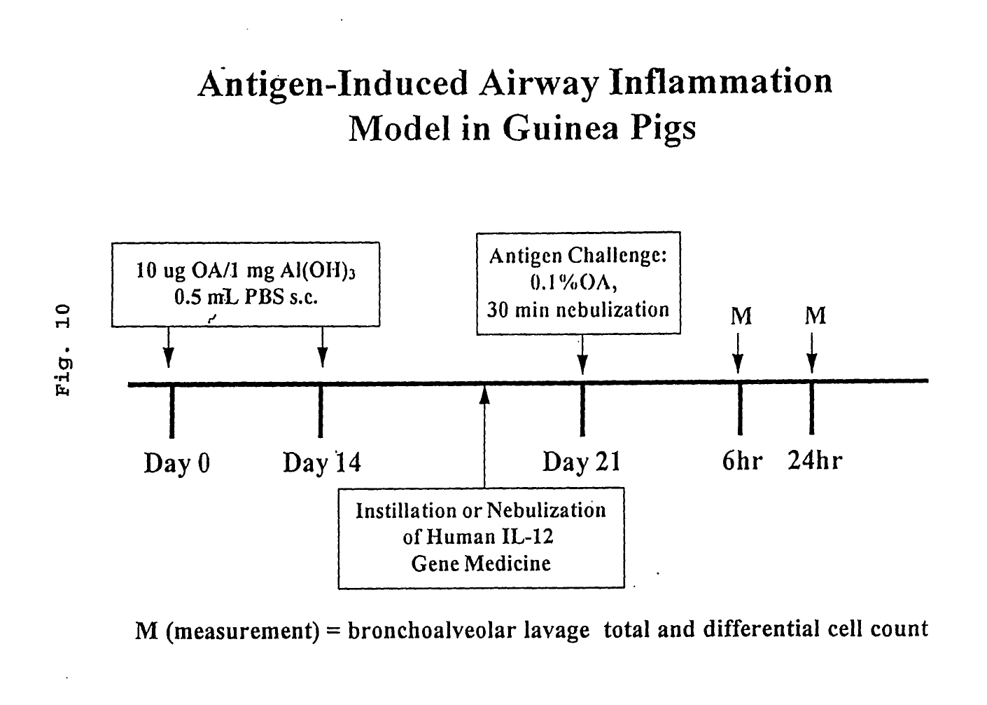

The effects of the administration of the IL-12 DNA:lipid formulations described above were evaluated using an antigen-induced airway inflammation model in guinea pigs. A timeline schematic of the model is shown in FIG. 10. In general the tests were performed using Hartley guinea pigs which were purchased at about 250-300 grams, and used at 450-500 grams body weight. The formulation was administered by instillation in a similar manner as described above for rats. The instilled formulation volume was 1 ml, which contained 50 μg of IL-12 encoding plasmid DNA, with DOTMA:chol in 10% lactose, and a negative to positive charge ratio of 1:3. The formulation was prepared using 800 nm extrusion size liposomes. At 6 hours (or 24 hours for some tests) after the ovalbumin (OA) antigen challenge on day 21, the animal was killed and the lungs lavaged with 3×10 mL Hank's solution to collect cells. The cells were removed from the lavage fluid by centrifugation, resuspended, the red blood cells lysed, and intact cells counted.

Using this guinea pig model of asthma, it was demonstrated that pretreatment of guinea pigs with a formulation containing 50 μg of the IL-12 encoding construct reduced the bronchioalveolar lavage cell (BAL) counts to a comparable extent as dexamethasone administered at 30 mg/kg one hour prior to the OA challenge and 4 hours post OA challenge. Those BAL cell counts were similar to the cell counts with no OA challenge. In comparison, guinea pigs which were challenged with OA, but not treated with any formulation, and guinea pigs which were challenged with OA following delivery of a formulation having a CAT encoding expression construct had similar, higher BAL cell counts. The formulation containing CAT encoding DNA did not reduce BAL cell counts.

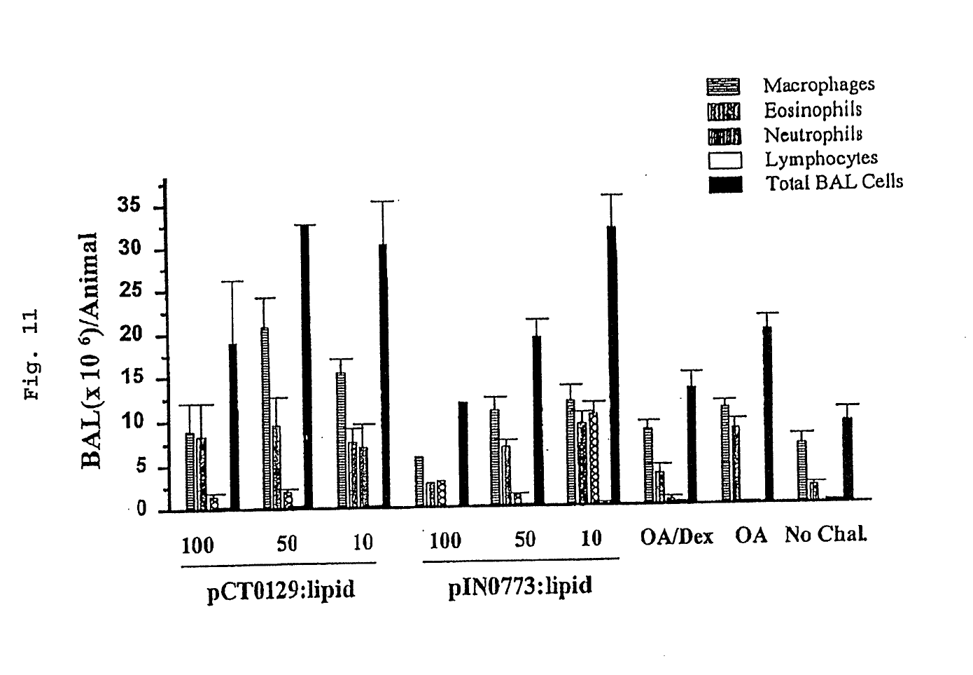

Consistent with IL-12's anti-inflammatory actions, not only is a marked reduction in total BAL cell number observed, but specifically the number of eosinophils in the BAL cells is reduced. See FIG. 11.

The concentration of human IL-12 in the lavage fluid was also determined; increasing IL-12 concentration was found to generally correlate with decreasing total BAL cell counts. The IL-12 concentrations were determined using sandwich ELISA assays for human IL-12 heterodimers (QUANTIKINE™ and QUANTIKINE™ High Sensitivity from R&D Systems). The selection of assay kit depended on expected IL-12 concentration in the fluid to be tested. These assays were shown to reliably quantitate human IL-12 in both lavage fluid and in cell extracts. Other assay methods can also be used for IL-12 quantitation, as similarly indicated for CAT assays.

The reduction in BAL cell counts in guinea pigs pretreated with the IL-12 encoding formulations was also shown to not be present when the animals were instead pretreated with various combinations of non-IL-12 encoding formulation components, or with a formulation containing CAT encoding DNA. In a set of tests the following BAL cell counts were observed for guinea pigs receiving the following treatments: (1) with no OA challenge, approximately 5×10⁶ BAL cells/animal; (2) OA challenge with dexamethasone treatment, approximately 9×10⁶ cells/animal; (3) OA challenge, approximately 28×10⁶ cells/animal; (4) OA challenge and 10% lactose pretreatment, approximately 41×10⁶ cells/animal; (5) OA challenge and pretreatment with 50 μg CAT encoding DNA in water, approximately 30×10⁶ cells/animal.

The results obtained with this model indicate that the administration of the formulations of this invention, containing IL-12 encoding DNA, to the lung produces biologically effective expression of IL-12.

### VI. Administration

Administration as used herein refers to the route of introduction of a plasmid or carrier of DNA into the body. In addition to the methods of delivery described above, the expression systems constructs and the delivery system formulations can be administered by a variety of different methods.

Administration can be directly to a target tissue or by targeted delivery to the target tissue after systemic administration. In particular, the present invention can be used for treating disease by administration of the expression system or formulation to the body in order to establishing controlled expression of any specific nucleic acid sequence within tissues at certain levels that are useful for gene therapy.

The preferred means for administration of vector and use of formulations for delivery are described above. The preferred embodiments are by deposit of a nebulized formulation into the airway of an animal or by direct injection using needle injection or hypospray.

The route of administration of any selected vector construct will depend on the particular use for the expression vectors. In general, a specific formulation for each vector construct used will focus on vector uptake with regard to the particular targeted tissue, followed by demonstration of efficacy. Uptake studies will include uptake assays to evaluate cellular uptake of the vectors and expression of the DNA of choice. Such assays will also determine the localization of the target DNA after uptake, and establishing the requirements for maintenance of steady-state concentrations of expressed protein. Efficacy and cytotoxicity can then be tested. Toxicity will not only include cell viability but also cell function.

Muscle cells have the unique ability to take up DNA from the extracellular space after simple injection of DNA particles as a solution, suspension, or colloid into the muscle. Expression of DNA by this method can be sustained for several months.

Delivery of formulated DNA vectors involves incorporating DNA into macromolecular complexes that undergo endocytosis by the target cell. Such complexes may include lipids, proteins, carbohydrates, synthetic organic compounds, or in-organic compounds. Preferably, the complex includes DNA, a cationic lipid, and a neutral lipid in particular proportions. The characteristics of the complex formed with the vector (size, charge, surface characteristics, composition) determines the bioavailability of the vector within the body. Other elements of the formulation function as ligand which interact with specific receptors on the surface or interior of the cell. Other elements of the formulation function to enhance entry into the cell, release from the endosome, and entry into the nucleus.

Delivery can also be through use of DNA transporters. DNA transporters refers to molecules which bind to DNA vectors and are capable of being taken up by epidermal cells. DNA transporters contain a molecular complex capable of noncovalently binding to DNA and efficiently transporting the DNA through the cell membrane. It is preferable that the transporter also transport the DNA through the nuclear membrane. See, e.g., the following applications all of which (including drawings) are hereby incorporated by reference herein: (1) Woo et al., U.S. Ser. No. 07/855,389, entitled “A DNA Transporter System and Method of Use,, filed Mar. 20, 1992, now abandoned; (2) Woo et al., PCT/US93/02725, International Publ. WO93/18759, entitled “A DNA Transporter System and method of Use”, (designating the U.S. and other countries) filed Mar. 19, 1993; (3) continuation-in-part application by Woo et al., entitled “Nucleic Acid Transporter Systems and Methods of Use”, filed Dec. 14, 1993, U.S. Ser. No. 08/167,641; (4) Szoka et al., U.S. Ser. No. 07/913,669, entitled “Self-Assembling Polynucleotide Delivery System”, filed Jul. 14, 1992 and (5) Szoka et al., PCT/US93/03406, International Publ. WO93/19768 entitled “Self-Assembling Polynucleotide Delivery System”, (designating the U.S. and other countries) filed Apr. 5, 1993.

Transfer of genes directly into muscle has been very effective. Experiments show that administration by direct injection of DNA into muscle cells results in expression of the gene in the area of injection. Injection of plasmids containing IGF-I results in expression of the gene for months at relatively constant levels. The injected DNA appears to persist in an unintegrated extrachromosomal state. This means of transfer is a preferred embodiment.

Another preferred method of delivery involves a DNA transporter system. The DNA transporter system consists of particles containing several elements that are independently and non-covalently bound to DNA. Each element consists of a ligand which recognizes specific receptors or other functional groups such as a protein complexed with a cationic group that binds to DNA. Examples of cations which may be used are spermine, spermine derivatives, histone, cationic peptides and/or polylysine. One element is capable of binding both to the DNA vector and to a cell surface receptor on the target cell. Examples of such elements are organic compounds which interact with the asialoglycoprotein receptor, the folate receptor, the mannose-6-phosphate receptor, or the carnitine receptor. A second element is capable of binding both to the DNA vector and to a receptor on the nuclear membrane. The nuclear ligand is capable of recognizing and transporting a transporter system through a nuclear membrane. An example of such ligand is the nuclear targeting sequence from SV40 large T antigen or histone. A third element is capable of binding to both the DNA vector and to elements which induce episomal lysis. Examples include inactivated virus particles such as adenovirus, peptides related to influenza virus hemagglutinin, or the GALA peptide described in the Szoka patent cited above.

Administration may also involve lipids as described in preferred embodiments above. The lipids may form liposomes which are hollow spherical vesicles composed of lipids arranged in unilamellar, bilamellar, or multi-lamellar fashion and an internal aqueous space for entrapping water soluble compounds, such as DNA, ranging in size from 0.05 to several microns in diameter. Lipids may be useful without forming liposomes. Specific examples include the use of cationic lipids and complexes containing DOPE which interact with DNA and with the membrane of the target cell to facilitate entry of DNA into the cell.

Gene delivery can also be performed by transplanting genetically engineered cells. For example, immature muscle cells called myoblasts may be used to carry genes into the muscle fibers. Myoblast genetically engineered to express recombinant human growth hormone can secrete the growth hormone into the animalls blood. Secretion of the incorporated gene can be sustained over periods up to 3 months.

Myoblasts eventually differentiate and fuse to existing muscle tissue. Because the cell is incorporated into an existing structure, it is not just tolerated but nurtured. Myoblasts can easily be obtained by taking muscle tissue from an individual who needs gene therapy and the genetically engineered cells can also be easily put back with out causing damage to the patient Is muscle. Similarly, keratinocytes may be used to delivery genes to tissues. Large numbers of keratinocytes can be generated by cultivation of a small biopsy. The cultures can be prepared as stratified sheets and when grafted to humans, generate epidermis which continues to improve in histotypic quality over many years. The keratinocytes are genetically engineered while in culture by transfecting the keratinocytes with the appropriate vector. Although keratinocytes are separated from the circulation by the basement membrane dividing the epidermis from the dermis, human keratinocytes secrete into circulation the protein produced.

The chosen method of delivery should result in expression of the gene product encoded within the nucleic acid cassette at levels which exert an appropriate biological effect. The rate of expression will depend upon the disease, the pharmacokinetics of the vector and gene product, and the route of administration, but should be between 1-1000 mg/kg of body weight/day. This level is readily determinable by standard methods. It could be more or less depending on the optimal dosing. The duration of treatment will extend through the course of the disease symptoms, possibly continuously. The number of doses will depend upon the disease, delivery vehicle, and efficacy data from clinical trials.

One skilled in the art will readily appreciate that the present invention is well adapted to obtain the ends and advantages mentioned as well as those inherent therein. The plasmid constructs described herein along with the formulations, methods, procedures, and treatments are presently representative of exemplary preferred embodiments, and are not intended as limitations on the scope of the invention. Changes therein and other uses will occur to those skilled in the art, and are encompassed within the spirit of the invention or defined by the scope of the claims.

It will be readily apparent to one skilled in the art that varying substitutions and modifications may be made to the invention disclosed herein without departing from the scope and spirit of the invention.

All patents and publications mentioned in the specification are herein incorporated by reference to the same extent as if each individual publication was specifically and individually indicated to be incorporated by reference.

What we claim is:

1. A plasmid for expression of recombinant eucaryotic genes comprising:
a first transcription unit comprising a first transcriptional control sequence transcriptionally linked with a first 5′-untranslated region, a first synthetic intron, a first coding sequence, and a first synthetic 3′-untranslated region/poly(A) signal, wherein said first synthetic intron is between said control sequence and said first coding sequence; and
a second transcription unit comprising a second transcriptional control sequence transcriptionally linked with a second 5′-untranslated region, a second synthetic intron, a second coding sequence, and a second synthetic 3′-untranslated region/poly(A) signal, wherein said second synthetic intron is between said control sequence and said second coding sequence.

2. The plasmid of claim 1, wherein said first transcriptional control sequence or said second transcriptional control sequence comprise cytomegalovirus promoter/enhancer sequences.

3. The plasmid of claim 1, wherein said first coding sequence or said second coding sequence encode a therapeutic molecule or a subunit of a therapeutic molecule.

4. The plasmid of claim 1, wherein said first and second transcriptional control sequences are the same.

5. The plasmid of claim 1, wherein said first and second transcriptional control sequences are different.

6. The plasmid of claim 1, wherein said first coding sequence and said second coding sequence comprise a sequence coding for the p40 subunit of human IL-12 and a sequence coding for the p35 subunit of human IL-12.

7. The plasmid of claim 6, wherein said sequence coding for the p40 subunit of human IL-12 is 5′ to said sequence coding for the p35 subunit of human IL-12.

8. A plasmid for expression of recombinant eucaryotic genes, comprising an intron having variable splicing, a first coding sequence, and a second coding sequence.

9. The plasmid of claim 8, further comprising:
a transcriptional control sequence transcriptionally linked with a first coding sequence and a second coding sequence;
a 5′-untranslated region;
an intron 5′ to said first coding sequence;
an alternative splice site 3′ to said first coding sequence and 5′ to said second coding sequence; and
a 3′-untranslated region/poly(A) signal.

10. The plasmid of claim 9, wherein said first coding sequence or said second coding sequence encode a therapeutic molecule or a subunit of a therapeutic molecule.

11. The plasmid of claim 9, wherein said transcriptional control sequence comprises a cytomegalovirus promoter/enhancer sequence.

12. The plasmid of claim 8, wherein said first coding sequence and said second coding sequence comprise a sequence coding for the p40 subunit of human IL-12 and a sequence coding for the p35 subunit of human IL-12.

13. A plasmid for expression of recombinant eucaryotic genes comprising:
a transcriptional control sequence transcriptionally linked with a first coding sequence, an IRES sequence, a second coding sequence, and a 3′-untranslated region/poly(A) signal, wherein said IRES sequence is between said first coding sequence and said second coding sequence; and
an intron between said promoter and said first coding sequence.

14. The plasmid of claim 13, wherein said transcriptional control sequence comprises a cytomegalovirus promoter/enhancer sequence.

15. The plasmid of claim 13, wherein said first coding sequence and said second coding sequence comprise a sequence coding for the p40 subunit of human IL-12 and a sequence coding for the p35 subunit of human IL-12.

16. The plasmid of claim 13, wherein said IRES sequence is from an encephalomyocarditis virus.

17. A DNA sequence coding for human IL-12 subunit, comprising a synthetic nucleotide sequence having less than 50% identity to a natural human IL-12 subunit coding sequence.

18. The DNA sequence of claim 17, wherein said synthetic nucleotide sequence comprises a sequence having at least 99% sequence identity to the sequence of SEQ ID NO. 3.

19. The DNA sequence of claim 18, wherein said synthetic nucleotide sequence comprises a nucleotide sequence identical to the sequence of SEQ ID NO. 3 or 4.

20. The DNA sequence of claim 17, wherein said synthetic nucleotide sequence comprises a sequence having at least 99% sequence identity to the sequence of SEQ ID NO. 7.

21. The DNA sequence of claim 20, wherein said synthetic nucleotide sequence comprises a nucleotide sequence identical to the sequence of SEQ ID NO. 7 or 8.

22. A composition for delivery of a DNA molecule in a mammal, comprising
a cationic lipid with a neutral co-lipid, prepared as a liposome having an extrusion size of about 800 nanometers; and
a quantity of DNA comprising a coding sequence.

23. The composition of claim 22, wherein said DNA is at least about 80% supercoiled.

24. The composition of claim 23, wherein said DNA is at least about 90% supercoiled.

25. The composition of claim 24, wherein said DNA is at least about 95% supercoiled.

26. The composition of claim 22, wherein said cationic lipid and said DNA are present in a negative to positive charge ratio of about 1:3.

27. The composition of claim 22, further comprising an isotonic carbohydrate solution.

28. The composition of claim 27, wherein said isotonic carbohydrate solution consists essentially of about 10% lactose.

29. A composition of claim 22, wherein said cationic lipid is DOTMA and said neutral co-lipid is cholesterol.

30. A composition for delivery of a DNA molecule in a mammal, comprising
a cationic lipid with a neutral co-lipid; and
a quantity of DNA comprising a coding sequence,
wherein said cationic lipid and said DNA are present in a negative to positive charge ratio of about 1:3.

31. The composition of claim 30, wherein said DNA is at least about 80% supercoiled.

32. The composition of claim 31, wherein said DNA is at least about 90% supercoiled.

33. The composition of claim 32, wherein said DNA is at least about 95% supercoiled.

34. The composition of claim 30, further comprising an isotonic carbohydrate solution.

35. The composition of claim 34, wherein said isotonic carbohydrate solution consists essentially of about 10% lactose.

36. A composition of claim 30, wherein said cationic lipid is DOTMA and said neutral co-lipid is cholesterol.

37. A method for preparing a composition for delivery of a DNA to a mammal, comprising the steps of:
a. preparing a DNA comprising a coding sequence;
b. preparing liposomes having an extrusion size of about 800 nm, wherein said liposomes comprise a cationic lipid and a neutral co-lipid; and
c. combining said liposomes with said DNA in amounts such that said cationic lipid and said DNA are present in a negative to positive charge ratio of about 1:3.

38. A method of treatment of a mammalian condition or disease, comprising administering to a mammal suffering from said condition or disease an amount of a composition for delivery of a DNA molecule in a mammal,
wherein said DNA comprises a coding sequence encoding a therapeutic molecule or a subunit thereof, and
wherein said composition comprises a cationic lipid, a neutral co-lipid, and said DNA, and has a negative to positive charge ratio of about 1:3 for said cationic lipid and said DNA.

39. The method of claim 38, wherein said composition is prepared for administration by ultrasonic nebulization.

40. The method of claim 38, wherein said DNA comprises two coding sequences which encode human IL-12 p40 and p35 subunits.

41. The method of claim 38, wherein said disease or condition is asthma.

42. The method of claim 38, wherein said disease or condition is a cancer.

43. A vaccine adjuvant comprising a cationic lipid, a neutral co-lipid, and DNA,
wherein said DNA comprises a sequence encoding the p40 subunit of IL-12 and the p35 subunit of IL-12, and
wherein said cationic lipid and said DNA are present in a negative to positive charge ratio of about 1:3.

44. A method of enhancing the response of a mammal to a vaccine, comprising the step of administering to said mammal a vaccine and an adjuvant,
wherein said adjuvant comprises a cationic lipid, a neutral co-lipid, and DNA, said DNA comprising a sequence encoding the p40 subunit of IL-12 and the p35 subunit of IL-12, and
wherein said cationic lipid and said DNA are present in a negative to positive charge ratio of about 1:3.
